SEQUENCE LISTING (51 sequences)

SEQ ID NO: 1 - protein, 6 aa, synthetic construct
HHHHHH

SEQ ID NO: 2 - RNA, 13 nt, synthetic construct
  source 1..13: mol_type = other RNA
aaaaaaaaaaaaa

SEQ ID NO: 3 - DNA, 35 bp, synthetic construct
  source 1..35: mol_type = other DNA
aagatcggaagagcacacgtctgaactccagtcac

SEQ ID NO: 4 - DNA, 34 bp, synthetic construct
  source 1..34: mol_type = other DNA
aagatcggaagagcgtcgtgtagggaaagagtgt

SEQ ID NO: 5 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 100 bp, synthetic construct
  source 1..100: mol_type = other DNA
tttttttttttttttttttttttttttttttttttttttttttttttttttttttttttttttttttttttttttttttttttttttttttttttttttt

SEQ ID NO: 7 - DNA, 20 bp, synthetic construct
  source 1..20: mol_type = other DNA
cttgtccgctacgttcgcca

SEQ ID NO: 8 - DNA, 20 bp, synthetic construct
  source 1..20: mol_type = other DNA
gagcttcatgacccaccaag

SEQ ID NO: 9 - DNA, 20 bp, synthetic construct
  source 1..20: mol_type = other DNA
gtactctagagagtaagtca

SEQ ID NO: 10 - DNA, 20 bp, synthetic construct
  source 1..20: mol_type = other DNA
tatctctgagccacattgag

SEQ ID NO: 11 - DNA, 20 bp, synthetic construct
  source 1..20: mol_type = other DNA
caatgtggctcagagatacg

SEQ ID NO: 12 - DNA, 20 bp, synthetic construct
  source 1..20: mol_type = other DNA
tgaggatagccaaggttctg

SEQ ID NO: 13 - DNA, 20 bp, synthetic construct
  source 1..20: mol_type = other DNA
agtcacatgtggtggaatgg

SEQ ID NO: 14 - DNA, 20 bp, synthetic construct
  source 1..20: mol_type = other DNA
gattgatcatgagcacagaa

SEQ ID NO: 15 - DNA, 20 bp, synthetic construct
  source 1..20: mol_type = other DNA
caggcagccgagatgcttta

SEQ ID NO: 16 - DNA, 20 bp, synthetic construct
  source 1..20: mol_type = other DNA
tctcggctgcctgctcaatc

SEQ ID NO: 17 - DNA, 20 bp, synthetic construct
  source 1..20: mol_type = other DNA
gggagcgaatcgccaacacg

SEQ ID NO: 18 - DNA, 20 bp, synthetic construct
  source 1..20: mol_type = other DNA
gcagcaggtctacaaccact

SEQ ID NO: 19 - DNA, 20 bp, synthetic construct
  source 1..20: mol_type = other DNA
gtccacaaacaggtcgtcgt

SEQ ID NO: 20 - DNA, 20 bp, synthetic construct
  source 1..20: mol_type = other DNA
tagctctcagcaactctttg

SEQ ID NO: 21 - DNA, 20 bp, synthetic construct
  source 1..20: mol_type = other DNA
cagttgaaggcacactgtaa

SEQ ID NO: 22 - DNA, 20 bp, synthetic construct
  source 1..20: mol_type = other DNA
aaaggctggcatgctgagca

SEQ ID NO: 23 - DNA, 20 bp, synthetic construct
  source 1..20: mol_type = other DNA
tttggacataacacacattg

SEQ ID NO: 24 - DNA, 20 bp, synthetic construct
  source 1..20: mol_type = other DNA
agcccatttagtccctgtct

SEQ ID NO: 25 - DNA, 20 bp, synthetic construct
  source 1..20: mol_type = other DNA
gtcacctgtgcctttgagca

SEQ ID NO: 26 - DNA, 20 bp, synthetic construct
  source 1..20: mol_type = other DNA
tctcatcgtgagatacattg

SEQ ID NO: 27 - DNA, 20 bp, synthetic construct
  source 1..20: mol_type = other DNA
tgtcctagagagttacagcc

SEQ ID NO: 28 - DNA, 20 bp, synthetic construct
  source 1..20: mol_type = other DNA
acagacagttcagaaagtga

SEQ ID NO: 29 - DNA, 20 bp, synthetic construct
  source 1..20: mol_type = other DNA
gatctgaaagccatcatcac

SEQ ID NO: 30 - DNA, 20 bp, synthetic construct
  source 1..20: mol_type = other DNA
tcataacatttgtcacagag

SEQ ID NO: 31 - DNA, 20 bp, synthetic construct
  source 1..20: mol_type = other DNA
ccatcggcaccaacctgcgc

SEQ ID NO: 32 - DNA, 20 bp, synthetic construct
  source 1..20: mol_type = other DNA
gagcttgtaccggaactccg

SEQ ID NO: 33 - DNA, 20 bp, synthetic construct
  source 1..20: mol_type = other DNA
cagccatggacaatcaggtg

SEQ ID NO: 34 - DNA, 20 bp, synthetic construct
  source 1..20: mol_type = other DNA
atggaagagcgtcatgactt

SEQ ID NO: 35 - DNA, 20 bp, synthetic construct
  source 1..20: mol_type = other DNA
ccagttgctctgggggaaca

SEQ ID NO: 36 - DNA, 20 bp, synthetic construct
  source 1..20: mol_type = other DNA
caccaaactcctgagcatca

SEQ ID NO: 37 - DNA, 20 bp, synthetic construct
  source 1..20: mol_type = other DNA
ctgagcatcacggtactcac

SEQ ID NO: 38 - DNA, 20 bp, synthetic construct
  source 1..20: mol_type = other DNA
acatgtcatgcaagcaccag

SEQ ID NO: 39 - DNA, 20 bp, synthetic construct
  source 1..20: mol_type = other DNA
tccatcatgccgatactgca

SEQ ID NO: 40 - DNA, 20 bp, synthetic construct
  source 1..20: mol_type = other DNA
cagcggaggtgattcactgg

SEQ ID NO: 41 - DNA, 20 bp, synthetic construct
  source 1..20: mol_type = other DNA
gctgtgcccacacaaagacg

SEQ ID NO: 42 - DNA, 20 bp, synthetic construct
  source 1..20: mol_type = other DNA
cgttggcaaattgcacctcg

SEQ ID NO: 43 - DNA, 20 bp, synthetic construct
  source 1..20: mol_type = other DNA
tgcaggtgaagctggagctg

SEQ ID NO: 44 - DNA, 20 bp, synthetic construct
  source 1..20: mol_type = other DNA
gacttccagtagctccaagg

SEQ ID NO: 45 - DNA, 20 bp, synthetic construct
  source 1..20: mol_type = other DNA
atgtgctgcgtatgaaagaa

SEQ ID NO: 46 - DNA, 20 bp, synthetic construct
  source 1..20: mol_type = other DNA
aaccgccaagactgatgcgg

SEQ ID NO: 47 - DNA, 20 bp, synthetic construct
  source 1..20: mol_type = other DNA
cacaaccgccaagactgatg

SEQ ID NO: 48 - DNA, 20 bp, synthetic construct
  source 1..20: mol_type = other DNA
agaggcctcgaagctttgtc

SEQ ID NO: 49 - DNA, 20 bp, synthetic construct
  source 1..20: mol_type = other DNA
gtcgagaagtgagcacaact

SEQ ID NO: 50 - DNA, 20 bp, synthetic construct
  source 1..20: mol_type = other DNA
ccctctggtcctgctgtgtc

SEQ ID NO: 51 - DNA, 20 bp, synthetic construct
  source 1..20: mol_type = other DNA
actgtaatcatctcaaaatt

(19) United States
(12) Patent Application Publication
(10) Pub. No.: US 2024/0401012 A1
(43) Pub. Date: Dec. 5, 2024

(54) SGF29 AS A TARGET FOR HOX/MYC DRIVEN CANCERS

(71) Applicant: Sanford Burnham Prebys Medical Discovery Institute, La Jolla, CA (US)

(72) Inventors: Aniruddha Jayant DESHPANDE, San Diego, CA (US); Anagha DESHPANDE, San Diego, CA (US); Karina Ofelia BARBOSA GUERRA, Tijuana (MX)

(21) Appl. No.: 18/435,802

(22) Filed: Feb. 7, 2024

Related U.S. Application Data
(60) Provisional application No. 63/593,640, filed on Oct. 27, 2023.
(60) Provisional application No. 63/483,712, filed on Feb. 7, 2023.

(51) Int. Cl. C12N 9/22; A61K 31/713; A61P 35/02; C07K 16/18; C12N 15/113; G01N 33/50

(52) U.S. Cl. CPC C12N 9/22; A61K 31/713; A61P 35/02; C07K 16/18; C12N 15/113; G01N 33/5011; C12N 2310/11; C12N 2310/122; C12N 2310/14; C12N 2310/20

(57) ABSTRACT

Described herein are methods and compositions for diagnosing, treating, or ameliorating symptoms of cancer, including Acute Myeloid Leukemia or acute lymphoblastic leukemia, with an inhibitor of SGF29.

### CROSS-REFERENCE TO RELATED APPLICATIONS

Any and all priority claims identified in the Application Data sheet, or any correction thereto, are hereby incorporated by reference under 37 CFR 1.57. For example, this application claims priority to U.S. Provisional Application No. 63/483,712, filed Feb. 7, 2023, and U.S. Provisional Application No. 63/593,640, filed Oct. 27, 2023, each of which are incorporated herein by reference in their entireties.

### STATEMENT AS TO FEDERALLY SPONSORED RESEARCH

This invention was made with government support under W81XWH-20-1-0703 awarded by the Medical Research and Development Command, and R01 CA262746 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Feb. 2, 2024, is named 42256-618_201_SL.xml and is 45,853 bytes in size.

### INCORPORATION BY REFERENCE

All publication, patents, and patent applications mentioned in this specification are herein incorporated by reference to the same extent as if each individual publication, patent, or patent application was specifically and individually indicated to be incorporated by reference. Additionally, this application is related to the following bioRxiv preprint publication www.biorxiv.org/content/10.1101/2022.12.12.519332v1, which became available on Dec. 13, 2022, and which is incorporated herein by reference in its entirety, and the following Blood research article 10.1182/blood.2023021234 (Reference 1), which became available on Dec. 4, 2023 and which is incorporated herein by reference in its entirety.

### BACKGROUND

Acute Myeloid Leukemia (AML) is a devastating form of blood cancer with a dismal survival rate. Current therapies are often associated with strong toxicity and undesirable side effects, highlighting the need for safer, more effective alternatives. A major challenge in the development of new drugs for AML is the heterogeneity of the disease at the molecular level. AML is composed of several morphologic and molecular subtypes, with distinct mutational profiles and clinical characteristics. However, there are molecular pathways that are commonly dysregulated across different AML subtypes. Studies have shown that a variety of upstream genetic alterations in AML, including DNMT3 mutations, NPM1 mutations and the gene fusion products of several different chromosomal translocations result in the activation of the clustered homeobox (HOX) genes and their co-factor proteins such as the three amino acid loop extension (TALE) homeobox gene MEIS1. Specific HOX genes such as the HOXA and/or HOXB genes as well as MEIS1 display significantly higher expression in a large proportion of AML samples compared to normal bone marrow cells. In particular, highly elevated expression of posterior HOXA or B genes and MEIS1 can be observed in as many as two-thirds of AML with diverse mutational profiles. Studies conducted using both loss-of-function as well as gain-of-function approaches have demonstrated the importance of HOX/MEIS expression in leukemia pathogenesis in diverse subsets of AML including those bearing MLL-fusions, NUP98-fusions, and AF10-fusions, to name a few. Importantly, ectopic HOXA9 overexpression in murine hematopoietic stem and progenitor cells (HSPCs) causes a myeloproliferative phenotype in mice that can progress to AML upon MEIS1-co-expression. MEIS1 is a critical co-factor of leukemia-associated HOXA transcription factors and is required for their full leukemogenic capability. MEIS1 has also been shown to act as a rate limiting regulator of leukemia stem cell (LSC) activity. Taken together, there is compelling evidence implicating the HOX/MEIS oncogenes as a key node integrating a variety of functionally distinct oncogenic insults in AML and possibly other leukemias.

Targeting of the HOX/MEIS pathway may yield therapeutic benefit in multiple, genetically heterogeneous AML sub-types. Since the HOX/MEIS proteins are DNA-binding transcription factors, they have proven difficult to target directly.

### SUMMARY OF THE INVENTION

Provided herein are methods and compositions for diagnosing, treatment, monitoring treatment, and selecting treatment of cancer. The disclosed methods and compositions are particularly suited for treatment of HOX/MYC driven cancers. In particular, the present disclosure reveals a novel role for SGF29 as a non-oncogenic dependency in AML and identifies the SGF29 Tudor domain as a novel, attractive therapeutic target in AML. SGF29 is shown herein to be an important regulator of the transcription as well as chromatin abundance of several leukemia-associated transcription factors in AML cells and plays an important role in sustaining the leukemogenesis of diverse AML oncoproteins. SGF29 deletion using CRISPR shows striking anti-leukemia activity in murine mouse models of AML, human cell lines and human patient derived xenografts in vitro and in vivo. Chromatin regulators such as SGF29 frequently possess druggable features and can be targeted as an indirect means to suppress the tumorigenic activity of dysregulated oncogenic transcription factors. In some embodiments, SGF29 may be targeted in AML patients with HOX/MEIS or MYC oncogene hyperactivation, which constitutes ˜70% of all AML. Also, SGF29 may play a wider role in cancers such as MYC-driven lymphomas, hepatocellular carcinoma, and other solid tumors. In some aspects, treatment comprises targeting SGF29 alone, or in combination with other epigenetic therapies and standard therapies for AML such as cytarabine, daunorubicin, etoposide, venetoclax, and/or azacytidine. In some aspects, treatment comprises targeting SGF29 alone, or in combination with other therapies that target HOX/MEIS or MYC oncogenes. In some aspects, SGF29 may be targeted in AML patients in order to prevent patient relapse. In some aspects, targeting SGF29 may reduce the number of leukemia stem cells.

In one aspect, the present disclosure provides a method of suppressing HOX/MEIS-activation in a cancer cell. In some embodiments, the method comprises obtaining a sample from a patient and determining if the patient has HOX/MEIS or MYC oncogene hyperactivation.

In some embodiments, the method further comprises treating cancer in a patient.

In some embodiments, the method further comprises administering a pharmaceutical composition for suppressing HOX/MEIS-activation in a cancer cell.

In some embodiments, the method further comprises treating Acute Myeloid Leukemia (AML) or Acute Lymphoblastic Leukemia (ALL) in a patient having a CALM-AF10 gene fusion mutation in a cell, the method comprising administering a pharmaceutical composition as disclosed and described herein to a subject in need thereof.

In some embodiments, the method further comprises preventing or reducing proliferation of a cell having a CALM-AF10 fusion gene mutation, the method comprising contacting the cell with (i) an inhibitor of SAGA-associated factor 29 (SGF29) protein, or (ii) an inhibitory agent of SAGA-associated factor 29 (SGF29) protein synthesis.

In some embodiments, the method further comprises treating or preventing Acute Myeloid Leukemia (AML) or Acute Lymphoblastic Leukemia (ALL) in a patient having a CALM-AF10 fusion gene mutation in a cell, comprising identifying a patient having a CALM-AF10 fusion gene mutation in a cell; and contacting the cell with (i) an inhibitor of SAGA-associated factor 29 (SGF29) protein, or (ii) an inhibitory agent of SAGA-associated factor 29 (SGF29) protein synthesis.

In some embodiments, the method further comprise identifying a test agent for treating or preventing Acute Myeloid Leukemia (AML) or Acute Lymphoblastic Leukemia (ALL) in a patient having a CALM-AF10 fusion gene mutation in a cell, comprising: contacting SAGA-Associated Factor 29 (SGF29) protein with a test agent; determining whether the test agent interacts with SGF29 protein; and subjecting the test agent to an in vitro, ex vivo or in vivo test, wherein a test agent that interacts with SGF29 protein reduces or prevents cell proliferation.

In some embodiments, the method further comprises identifying a test agent for treating or preventing Acute Myeloid Leukemia (AML) or Acute Lymphoblastic Leukemia (ALL) in a patient having a CALM-AF10 fusion gene mutation in a cell, comprising subjecting a test agent to an in vitro, ex vivo or in vivo model of AML or ALL cell growth, wherein the test agent interacts with SAGA-Associated Factor 29 (SGF29) protein in a cell.

In some embodiments, the method further comprises identifying a test agent for treating or preventing Acute Myeloid Leukemia (AML) or Acute Lymphoblastic Leukemia (ALL) in a patient having a CALM-AF10 fusion gene mutation in a cell, comprising: contacting a mammalian cell that expresses SAGA-Associated Factor 29 (SGF29) protein with a test agent; determining the activity of SGF29 protein in the cell relative to a similar cell that has not been contacted with the test agent; and identifying the test agent as a test agent that reduces cell proliferation, if the activity of SGF29 protein in step (b) is reduced in the cell that has been contacted with the test agent relative the similar cell that has not been contacted with the test agent.

In some embodiments, the method further comprises treating or preventing Acute Myeloid Leukemia (AML) or Acute Lymphoblastic Leukemia (ALL) in a patient having a CALM-AF10 fusion gene mutation in a cell, comprising: contacting a cell comprising SAGA-Associated Factor 29 (SGF29) protein operably linked to a reporter gene with a test agent; determining the level of expression of the reporter gene; and subjecting the test agent that reduces the level of expression of the reporter gene to an in vitro, ex vivo or in vivo test.

In some embodiments, the method further comprises screening a compound library to identify compounds which bind to SAGA-Associated Factor 29 (SGF29) protein for treating or preventing Acute Myeloid Leukemia (AML) or Acute Lymphoblastic Leukemia (ALL) in a patient having a CALM-AF10 fusion gene mutation in a cell, comprising: obtaining a library comprising a plurality of compound structures; performing the method of claim 64 with compounds from the compound library; obtaining a mean output value; and ordering the compounds based on the output value.

In some embodiments, the method further comprises screening a compound library to identify compounds which bind to SAGA-Associated Factor 29 (SGF29) protein for treating or preventing Acute Myeloid Leukemia (AML) or Acute Lymphoblastic Leukemia (ALL) in a patient having a CALM-AF10 fusion gene mutation in a cell, comprising: obtaining a library comprising a plurality of compound structures; obtaining a first SGF29 protein crystal structure and a second SGF29 protein crystal structure; performing molecular dynamic simulations on the first SGF29 protein crystal structure and the second SGF29 protein crystal structure; generating a SGF29 protein structure using RMSD (root-mean-square-difference) clustering of related residues from the molecular dynamic simulations; docking compounds to the SGF29 protein structure from step (d); and identifying compounds which bind to the SGF29 protein structure with a desired affinity.

In some embodiments, the method further comprises contacting a UB3 cell with (i) an inhibitor of SAGA-associated factor 29 (SGF29) protein, or (ii) an inhibitory agent of SAGA-associated factor 29 (SGF29) protein synthesis; and measuring HOX/MEIS expression.

In another aspect, a method for identifying a test agent for treating or preventing Acute Myeloid Leukemia (AML) or Acute Lymphoblastic Leukemia (ALL) in a patient having an elevated expression of HOX/MEIS or MYC oncogenes in a cell, comprising: contacting a SAGA-Associated Factor 29 (SGF29) protein with a test agent; determining whether the test agent interacts with a SGF29 protein; and subjecting the test agent to an in vitro, an ex vivo, or an in vivo test, wherein the test agent that interacts with a SGF29 protein reduces or prevents cell proliferation.

In some embodiments, the patient has a cell comprising a nucleophosmin (NPM1) mutation. In some embodiments, the cancer cell is an Acute Myeloid Leukemia (AML) cell, an Acute Lymphoblastic Leukemia (ALL) cell, a multiple myeloma cell, a rhabdomyosarcoma cell, a breast cancer cell, a lymphoma cell, or a skin cancer. In some embodiments, the cancer cell is CALM-AF10 fusion gene positive, MLL-AF9 fusion gene positive, an MLL/KMT2A rearranged AML cell, an NPM1 mutant AML cell, an AF10-rearranged cell, an AML1-ETO positive cancer cell, or a PML-RARA positive leukemia cell.

In another aspect, the present disclosure provides A method of treating or preventing multiple myeloma, rhabdomyosarcoma, breast cancer, lymphoma, or skin cancer in a patient having a CALM-AF10 fusion gene mutation in a cell, comprising identifying a patient having HOX/MEIS or MYC oncogene hyperactivation; and contacting the cell with (i) an inhibitor of SAGA-associated factor 29 (SGF29) protein, or (ii) an inhibitory agent of SAGA-associated factor 29 (SGF29) protein synthesis. In some embodiments, the inhibitor of SGF29 protein is a small molecule or an antibody. In some embodiments, the small molecule or antibody interacts with a Tudor domain of SGF29 protein. In some embodiments, the inhibitory agent of SGF29 protein synthesis is (i) a small interfering RNA (siRNA), (ii) a short hairpin RNA (shRNA), or (iii) an antisense oligonucleotide (ASO) that is complementary to an mRNA sequence. In some embodiments, the inhibitory agent of SGF29 protein synthesis is a siRNA. In some embodiments, the inhibitory agent of SGF29 protein synthesis is a shRNA. In some embodiments, the inhibitory agent of SGF29 protein synthesis is an ASO. In some embodiments, the siRNA hybridizes with an mRNA. In some embodiments, the inhibitory agent of a SGF29 protein synthesis is a CRISPR-Cas9 complex comprising (i) a Cas9 protein or a polynucleotide encoding the Cas9 protein, and (ii) a guide RNA or a polynucleotide encoding the guide RNA, wherein the guide RNA hybridizes with a nucleic acid sequence. the nucleic acid sequence is a DNA sequence. In some embodiments, the inhibitory agent of a SGF29 protein synthesis is a Transcription Activator-like Effector Nuclease (TALEN) or a polynucleotide encoding the TALEN. In some embodiments, the method further comprises obtaining the cell from the patient.

### BRIEF DESCRIPTION OF THE DRAWINGS

This patent application contains at least one drawing executed in color. Copies of this patent or patent application with color drawing(s) will be provided by the Office upon request and payment of the necessary fee.

FIGS. 1A-1F show that screening for epigenetic modulators identifies novel MEIS1 regulators in AML cells, in accordance with some embodiments described herein. FIG. 1A shows GFP-MEIS1 signal in U937 GFP-MEIS1 AML upon DOT1L CRISPR knockout plotted on the y-axis. Biological duplicates indicate independent sgRNAs. The y-axis indicates eGFP intensity values from single cells (n≥5,000). FIG. 1B shows a small-molecule screen in human U937 GFP-MEIS1 AML cells; y-axis indicates the percentage GFP-Meis1 intensity compared to controls on day 5 of treatment with the small molecule library; x-axis indicates a compound number assigned to list the compounds in the library. FIG. 1C shows bubble plots to illustrate the categories of chromatin modulators included in the library design; the numbers indicate the different genes under the category and the size of the bubble is proportional to the gene-set size. Controls refer to pan-essential genes. FIG. 1D shows strategy for phenotypic pooled CRISPR screening of epigenetic regulators for MEIS1 expression in the U937 cell line with an eGFP-MEIS1 endogenous knock-in tag. FIG. 1E shows sorted gene hits based on differential beta scores for the eGFP-MEIS1 low minus eGFP-MEIS1 high fractions. Beta scores were calculated using MAGeCKFlute. FIG. 1F shows annotated protein complexes comprising top candidate hits identified in the epigenetic screen as identified using String database analysis are shown. Hits from the epigenetics CRISPR library screen are marked with a glowing hue within the protein complex.

FIGS. 2A-2F show evidence of the requirement for MEIS1 regulators for transcriptomic changes and AML cell growth, in accordance with some embodiments described herein. FIG. 2A shows schematic of competition assay with cells transduced with sgRNAs expressed in a BFP+ backbone. The proportion of BFP-positive cells is assessed over time using flow cytometry. FIG. 2B shows the y-axis indicates flow cytometry measurements of the percentage of U937 BFP+ cells over time, normalized to the baseline (time 0 or T″0″) measurement (n=3 technical replicates); the x-axis indicates the sgRNA number for non-targeting controls (NTC) or each gene (n=2). Each bar represents a timepoint measurement: baseline, day 8, day 11, and day 14. FIG. 2C shows ridge plots for CROP-Seq perturbations. Knockout genes are indicated on the y-axis and RNA expression levels for the self-renewal-associated genes listed are on the x-axis. The median expression value is indicated. FIG. 2D shows ridge plots for CROP-Seq perturbations. Knockout genes are indicated on the y-axis and RNA expression levels for the differentiation-associated genes listed are on the x-axis. The median expression value is indicated. FIG. 2E shows a relative score of DepMap dependencies for AML (n=26) compared to non-AML cell lines (n=1080) is shown for candidate hits identified in our screen. Y-axis is the rank of AML selectivity, and the x-axis shows the negative log 10 p-values. FIG. 2F shows a sigmoid plot of DepMap data showing the dependency score (Chronos—x-axis), compared to the normalized dependency rank (y-axis) for SFG29 in 1,086 cancer cell lines.

FIGS. 3A-3F depict proliferative and transcriptional effects of SGF29 loss in AML, in accordance with some embodiments described herein. FIG. 3A shows retention of the CellTrace™ Far Red dye in U937 transduced with an SGF229 sgRNA (top) compared to non-targeting control (NTC), measured by flow cytometry over time. FIG. 3B shows retention of the CellTrace™ Far Red dye in MOLM13 cells transduced with an SGF229 sgRNA (top) compared to non-targeting control (NTC), measured by flow cytometry over time. FIG. 3C shows schematic for differentiation assessment via phagocytosis of fluorescently labeled E. coli bioparticles. FIG. 3D: Histograms of U937 cells transduced with an NTC (grey) or an SGF29 sgRNA (violet) show the fluorescence intensity of engulfed pHrodo™ Red E. coli bioparticles measured at 9 days after staining. Effect of SGF29 deletion on the transcription of HOXA/MEIS genes in FIG. 3E U937 cell line, compared to non-targeting control (NTC) is shown. TPM: transcripts per million, P val. *<0.05, **<0.01, ***<0.005. Effect of SGF29 deletion on the transcription of HOXA/MEIS genes in FIG. 3G MOLM13 AML cell line, compared to non-targeting control (NTC) is shown. TPM: transcripts per million, P val. *<0.05, **<0.01, ***<0.005. FIG. 3F shows Kaplan-Meier curves for NTC versus SGF29 knockout in U937 cell line and an MLL-AF10 patient-derived xenograft line. FIG. 3H shows Kaplan-Meier curves for NTC versus SGF29 knockout in MOLM13 AML cell lines and an MLL-AF10 patient-derived xenograft line.

FIGS. 4A-4K show Sgf29 deletion impairs the clonogenicity of transformed but not normal hematopoietic cells, in accordance with some embodiments provided herein. FIG. 4A: Number of colony-forming units (CFU) from CALM-AF10 transformed cells transduced with Sgf29 intron-targeting sgRNA (Sgf29-Int) or two independent Sgf29 exon-targeting sgRNAs are shown. CFUs per 2,000 plated cells at 1 week are plotted on the Y-axis, and colonies are divided into those with a blast-like or a differentiated colony morphology. FIG. 4B shows pictures of representative colonies with each of the labelled sgRNA-transduced cells of the CALM-AF10 transformed cells. Scale bar: 100 mm. FIG. 4C shows Wright-Giemsa stained cytospins of representative cells from each of the CRISPR perturbations of the CALM-AF10 transformed cells. Scale bar: 10 mm. FIG. 4D shows number of colony-forming units (CFU) from MLL-AF9-transformed BM cells transduced with Sgf29 intron-targeting sgRNA (Sgf29-Int) or two independent Sgf29 exon-targeting sgRNAs. CFUs per 2,000 plated cells at 1 week are plotted on the Y-axis, and colonies are divided into those with a blast-like or a differentiated colony morphology. FIG. 4E shows pictures of representative colonies with each of the labelled sgRNA-transduced cells of the MLL-AF9-transformed BM cells. Scale bar: 100 mm. FIG. 4F shows Wright-Giemsa stained cytospins of representative cells from each of the CRISPR perturbations of the MLL-AF9-transformed BM cells. Scale bar: 10 mm. FIG. 4G shows number of colony-forming units (CFU) from MLLAF10-transformed cells transduced with Sgf29 intron-targeting sgRNA (Sgf29-Int) or two independent Sgf29 exon-targeting sgRNAs. CFUs per 2,000 plated cells at 1 week are plotted on the Y-axis, and colonies are divided into those with a blast-like or a differentiated colony morphology. FIG. 4H shows pictures of representative colonies with each of the labelled sgRNA-transduced cells of the MLLAF10-transformed cells. Scale bar: 100 mm. FIG. 4I shows Wright-Giemsa stained cytospins of representative cells from each of the CRISPR perturbations of the MLLAF10-transformed cells. Scale bar: 10 mm. FIG. 4J shows CFUs per 10,000 lineage negative, Sca-1 positive, Kit positive (LSK) cells with Sgf29 deletion (Sgf_sg1) compared to Sgf29 wildtype LSKs (nontargeting control; NTC). Y-axis shows the numbers of different types of colonies, with each plot indicating a separate kind of morphologically distinct CFU in this assay. P values: *=p<0.05, **=P<0.01. FIG. 4K shows pictures of colonies observed in CFU assays.

FIGS. 5A-5D show evaluation of the Tudor domain of SGF29 activity in AML cells, in accordance with some embodiments provided herein. FIG. 5A: Proportion of SGF29 peaks in promoter regions and intergenic regions in U937 cells are shown in the Donut plot. FIG. 5B: SGF29 peaks in promoter regions and their associated with H3K4me3, and intergenic, intronic and other regions marked by H3K27ac in U937 cells are shown in the bar graph as marked. X-axis shows number of peaks. FIG. 5C: Genome tracks depicting normalized reads (y-axis) for wildtype or mutant SGF29 at the genomic locus of the HOXA/MEIS1, MYC, and BMI1 genes in U937 cells are shown. FIG. 5D: Top panel: Representative images of CFUs from MLL-AF9 transformed bone marrow cells with a mock transduction (two top left colonies), non-targeting control (two top middle colonies) or Sgf29 sgRNA (two right colonies) are shown. Middle panel: Same cells as above, but with ectopic retroviral overexpression of wildtype human SGF29 impervious to SGF29 sgRNA. Lower panel: Same MLL-AF9 transformed cells as in top panel but with retroviral overexpression of the human SGF29D196R mutant is shown. Representative colonies are shown in bright field at 20× magnification.

FIGS. 6A-6E show chromatin enrichment proteomics reveals SGF29-dependent proteins in the chromatin proteome, in accordance with some embodiments provided herein. FIG. 6A: Schematic for the ChEP sample preparation. FIG. 6B: A donut plot showing the functional protein categories in proteomic analysis of the ChEP SGF29-deleted fraction across 4 samples. Proteins were annotated. The number of proteins per category is shown in parentheses. FIG. 6C: Volcano plots depicting −log(10) of the adjusted P value on the y-axis and log(2) of fold change (LogFC) of all proteins in the ChEP fractions for both SGF29 knockout vs. NTC. Differentially abundant protein (Absolute FC>2, Adj. P value.<0.05 are not shown; n=3 for every experimental condition. FIG. 6D: Bar graphs depicting intensity values for self-renewal-associated proteins enriched by ChEP in the SGF29 knockout vs NTC experiment. FIG. 6E: Dot blots for ChEP-enriched chromatin, cytoplasmic fraction, and whole-cell lysate for NTC and SGF29 knockout are shown. Vinculin and Histone H3 were included as cytoplasmic and chromatin protein controls respectively.

FIGS. 7A-7E depict Effect on H3K9 acetylation and antileukemia activity of SGF29 in AML patient cells, in accordance with some embodiments provided herein. FIG. 7A: Volcano plot indicating changes in H3K9 acetylation across the genome in UB3 cells after deletion of SGF29. Blue dots are the loci with decreased and red are the loci with increased acetylation. FIG. 7B: Integrated genome viewer (IGV) tracks showing ChIP-seq peaks for H3K9ac on AML oncogene loci in U937 cells expressing non-targeting (grey), compared to SGF29 targeting sgRNAs (blue). FIG. 7C: In vitro competition assay is shown with percentage of SGF29 sgRNA expressing BFP positive AML393 human AML cells (y-axis) is shown in blue bars, compared to control cells in grey. Progressive percentage of BFP is shown over time at indicated timepoints shown on the y-axis. FIG. 7D: Relative expression of HOXA9 and MEIS1 in SGF29 deleted AML393 cells is plotted on the y-axis normalized to the SGF29 wildtype control (n=3). P values: **=p<0.05, ***=P<0.01. FIG. 7E: Kaplan-Meier curves for NTC versus SGF29 deleted AML393 PDX (MLL-AF10 positive) are shown with the survival probability plotted on the y-axis.

FIGS. 8A-D depict results from high-density, domain focused pooled CRISPR library screen identifies epigenetic regulators of MEIS1 expression, in accordance with some embodiments provided herein. FIG. 8A (S1A): Expression of indicated genes (MEIS1, HOXA9 and BMI1) in 42 AML cell lines from the DepMap data ranked by expression (x-axis is rank, high to low expression), (y-axis is log transformed transcripts per million (TPM)+1) values. FIG. 8B (S1B): Schematic for generation of inducible Tet-Off MLL-AF9 mouse AML in the GFPMeis1 background. GFP fluorescence intensity measured upon treatment with DMSO, DOT1L inhibitor, or doxycycline in mouse Tet-Off MLL-AF9 AML (n=3). FIG. 8C (S1C): Schematic for phenotypic epigenetics compound screen. FIG. 8D (S1D): Screen results for Tet-Off mouse MLL-AF9 AML; the x-axis indicates % viability on day 5 after treatment with small molecules; the y-axis indicates the percentage of decrease in GFP-Meis1 signal compared to controls. Doxycycline was a positive control that shut off MLL-AF9 expression.

FIGS. 9A-9C, FIGS. 10A-10C, FIGS. 11A-11B, and FIG. 12 depict further results from high-density, domain focused pooled CRISPR library screen identifies epigenetic regulators of MEIS1 expression, in accordance with some embodiments provided herein. FIG. 9A (S2A): sgRNA normalized counts in each sorted fraction (low vs high). Panels depict counts for non-targeting controls and polymerase essential genes, as well as the known HOX/MEIS regulators DOT1L and ENL/MLLT1. FIG. 9B (S2B): Violin plots show the GFP-MEIS fluorescence intensity values plotted on the y-axis from 2-3 unique sgRNAs targeting the indicated genes, compared to 2 non-targeting controls (NTC), measured 7 days after puromycin selection. FIG. 9C (S2C): MOLM13 BFP+ cells over time, normalized to the baseline measurement; the x-axis indicates the sgRNA number for non-targeting controls (NTC) or each gene (n=3). Each bar represents a timepoint measurement: baseline, day 8, day 11, and day 14 for U937; baseline, day 6, day 12, day 18, and day 24 for MOLM13. FIG. 10A (S2D): Uniform Manifold Approximation (UMAP) plots of synthetic gene expression profiles from CROP-Seq perturbations in U937 GFP-MEIS1 cells. FIGS. 10B-C: Hierarchical clustering dendrogram for gene expression of variable features: all genes (FIG. 10B (S2E)) or HOXA-cluster genes and MEIS1 (FIG. 10C (S2F)). Branches corresponding to perturbed genes by CRISPR. Fig. S2G: Ridge plots for CROP-Seq perturbations. Knockout genes are indicated on the y-axis and RNA expression levels for the (FIG. 11A (S2G)) self-renewal-associated genes or (FIG. 11B (S2H)) differentiation-associated genes are on the x-axis. The median expression value is indicated. FIG. 12 (S2I): Selectivity (y-axis) and efficacy (x-axis) analysis for candidate hits in the DepMap data is shown in the scatter plot. PSME4 and BRD4 are included as examples of genes with high efficacy and low selectivity. Each dot in the plot is a gene from the DepMap data. More negative efficacy values indicate higher dependence or fitness score and higher values on the selectivity plot indicates higher differences between fitness scores of sensitive and insensitive cell lines.

FIG. 13 and FIGS. 14A-14C_(S3A-S3D) shows SGF29 deletion has pronounced anti-leukemia effects in cell-line-derived and patient-derived AML models, in accordance with some embodiments provided herein. FIG. 13 (S3A): Western blotting for SGF29 in non-targeting control treated MOLM13 and U937 lysates compared to vinculin is shown as a loading control. FIG. 14A (S3B): Cell cycle progression analysis by propidium iodide staining in human AML cells expressing NTC (blue) or SGF29 (light violet) sgRNAs (n=3). FIG. 14B (S3C): Differences in MYC transcript levels are shown as TPMs on the y-axis in U937 and MOLM13 cells with SGF29 deletion. FIG. 14C (S3D): Hallmarks gene set enrichment analysis (GSEA) for the U937 cell line with SGF29 deletion compared to non-targeting control is shown.

FIGS. 15A-15E (S4A-S4E) shows Genetic Sgf29 inactivation impairs the clonogenicity of transformed but not normal hematopoietic progenitors, in accordance with some embodiments provided herein. FIG. 15A (S4A): Western blot demonstrating loss of SGF29 protein in murine CALM-AF10 leukemia cells transduced with SGF29 sgRNA1 or sgRNA2. Beta Actin as a loading control. FIG. 15B (S4B): Colony forming unit (CFU) assay in murine CALM-AF10 leukemia. 2nd week of CFU assay showing number of Blast-like as well as differentiated type colonies in cells transduced with intron targeting control or SGF29 targeting sgRNAs. Gray bar indicates cells with Intron whereas Blue bars indicate Sgf29 sgRNA expressing cells. (n=3); ns=non-significant; *=P<0.05. FIG. 15C (S4C): Colony forming unit (CFU) assay in murine MLL-AF9 leukemia cells showing second (left panel) and third (right panel) replating. Gray bars indicate the colonies from cells expressing Intron sgRNA. Blue bars indicate colonies from cells expressing sgRNAs targeting Sgf29. N=3); *=P<0.05; **=P<0.005. FIG. 15D (S4D): Colony forming unit (CFU) assay in murine MLL-AF10 transformed leukemia. The left panel indicates 2nd week and the right panel indicates 3rd week of the assay. Gray bars indicate the colonies from cells expressing Intron sgRNA. Blue bars indicate colonies from cells expressing sgRNAs targeting Sgf29. n=3); ns=non-significant; *=P<0.05; **=P<0.005; ***=P<0.0005. FIG. 15E (S4E): Effect of Sgf29 deletion on apoptosis in murine MLL-AF9 transformed leukemia. Immunoblot for PARP protein cleavage showing apoptosis in murine leukemia expressing Intron or Sgf29 targeting sgRNA. Two representative replicates are shown.

FIGS. 16A and 16B(S5A-S5B) shows evidence of the role of SGF29 in chromatin localization of proteins with established roles in AML pathogenesis, in accordance with some embodiments provided herein. FIG. 16A (S5A): SDS-PAGE showing whole cell extract, cytoplasmic and ChEP-enriched fractions for NTC vs SGF29 knockout. FIG. 16B (S5B): Heatmap of intensity changes in peptides for proteins evicted from the chromatin fraction of UB3 cells after SGF29 deletion in ChEP assay. R1, r2 and R3 are three independent replicates.

FIG. 17 (S6A) shows SGF29 deletion diminishes H3K9Ac on the promoters of key leukemia oncogenes, in accordance with some embodiments provided herein. FIG. 17 (S6A): Heatmaps showing histone H3K9 acetylation (H3K9Ac) peaks centered around transcription start site (TSS) in UB3 cells transduced with non-targeting control (NTC) or SGF29 targeting sgRNA.

FIG. 18 (S7A) shows cell cycle analysis in MLL-AF10 PDX cells after deletion of SGF29, in accordance with some embodiments provided herein. A representative sample showing changes in different phases of cell cycle upon loss of SGF29 is shown here. Orange represents cells expressing non-targeting control (NTC) sgRNA whereas blue indicates the cells with SGF29 ko.

### DETAILED DESCRIPTION

Some specific details of this description are set forth in order to provide a thorough understanding of various embodiments. However, one skilled in the art will understand that the present disclosure may be practiced without these details. In other instances, well-known structures and/or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All references cited herein are incorporated by reference in their entirety as though fully set forth. Singleton et al., Dictionary of Microbiology and Molecular Biology 3rd ed., J. Wiley & Sons (New York, NY 2001); March, Advanced Organic Chemistry Reactions, Mechanisms and Structure 5th ed., J. Wiley & Sons (New York, NY 2001); and Sambrook and Russel, Molecular Cloning: A Laboratory Manual 3rd ed., Cold Spring Harbor Laboratory Press (Cold Spring Harbor, NY 2001), provide one skilled in the art with a general guide to many of the terms used in the present application.

### Definitions

When indicating the number of substituents, the term “one or more” refers to the range from one substituent to the highest possible number of substitutions (e.g., replacement of one hydrogen up to replacement of all hydrogens by substituents).

The term “optional” or “optionally” denotes that a subsequently described event or circumstance can but need not occur, and that the description includes instances where the event or circumstance occurs and instances in which it does not.

The term “nucleic acid” as used herein generally refers to one or more nucleobases, nucleosides, or nucleotides, and the term includes polynucleobases, polynucleosides, and polynucleotides.

The term “polynucleotide”, as used herein generally refers to a molecule comprising two or more linked nucleic acid subunits (e.g., nucleotides, and can be used interchangeably with “oligonucleotide”). For example, a polynucleotide may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides include nucleotides in which the sugar is ribose. Deoxyribonucleotides include nucleotides in which the sugar is deoxyribose. A nucleotide can be a nucleoside monophosphate, nucleoside diphosphate, nucleoside triphosphate or a nucleoside polyphosphate. For example, a nucleotide can be a deoxyribonucleoside polyphosphate, such as a deoxyribonucleoside triphosphate (dNTP), Exemplary dNTPs include deoxyadenosine triphosphate (dATP), deoxycytidine triphosphate (dCTP), deoxyguanosine triphosphate (dGTP), uridine triphosphate (dUTP) and deoxythymidine triphosphate (dTTP). dNTPs can also include detectable tags, such as luminescent tags or markers (e.g., fluorophores). For example, a nucleotide can be a purine (e.g., A or G, or variant thereof) or a pyrimidine (e.g., C, T or U, or variant thereof). In some examples, a polynucleotide is deoxyribonucleic acid (DNA), ribonucleic acid (RNA), or derivatives or variants thereof. Exemplary polynucleotides include, but are not limited to, short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), and heteronuclear RNA (hnRNA), and encompasses both the nucleotide sequence and any structural embodiments thereof, such as single-stranded, double-stranded, triple-stranded, helical, hairpin, stem loop, bulge, etc. In some cases, a polynucleotide is circular. A polynucleotide can have various lengths. For example, a polynucleotide can have a length of at least about 7 bases, 8 bases, 9 bases, 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. For example, polynucleotide sequences may comprise isolated and purified DNA/RNA molecules, synthetic DNA/RNA molecules, and/or synthetic DNA/RNA analogs.

Polynucleotides may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

As used herein, the terms “polypeptide”, “protein” and “peptide” are used interchangeably and refer to a polymer of amino acid residues linked via peptide bonds, which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order. For example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein. For example, the portion of the protein may be a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant.

The terms “administer,” “administering”, “administration,” and the like, as used herein, refer to the methods that may be used to enable delivery of compounds or compositions to the desired site of biological action. These methods include, but are not limited to oral routes (p.o.), intraduodenal routes (i.d.), parenteral injection (including intravenous (i.v.), subcutaneous (s.c.), intraperitoneal (i.p.), intramuscular (i.m.), intravascular or infusion (inf.)), topical (top.) and rectal (p.r.) administration. Those of skill in the art are familiar with administration techniques that can be employed with the compounds and methods described herein. In some embodiments, the compounds and compositions described herein are administered orally.

The terms “effective amount” or “therapeutically effective amount,” as used herein, refer to a sufficient amount of an agent or a compound being administered which will relieve to some extent one or more of the symptoms of the disease or condition being treated. For example, a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The term “enhance” as used herein, means to increase or prolong either in amount, potency or duration a desired effect.

The terms “inhibitor” or “inhibitory agent” as used herein encompass compositions, agents, and compounds that inhibit expression or activity of a gene or protein. “Inhibit,” “inhibiting,” and “inhibition” and like terms include decreasing an activity, response, condition, disease, or other biological parameter. This can include but is not limited to the complete ablation of the expression, activity, response, condition, or disease. This may include, for example, a 10% reduction in the expression, activity, response, condition, or disease as compared to the native or control level. Thus, the reduction can be a 10, 20, 30, 40, 50, 60, 70, 80, 90, 100%, or any amount of reduction in between as compared to native or control levels.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a non-human animal is a mouse.

The terms “treat,” “treating” or “treatment,” as used herein, include alleviating, abating or ameliorating at least one symptom of a disease or condition, preventing additional symptoms, or inhibiting the disease or condition. Non-limiting examples of inhibiting the disease or condition include arresting the development of the disease or condition, relieving the disease or condition, causing regression of the disease or condition, relieving a condition caused by the disease or condition, or stopping the symptoms of the disease or condition either prophylactically and/or therapeutically.

The terms “pharmaceutical composition” and “pharmaceutical formulation” (or “formulation”) are used interchangeably. In some cases, the terms denote a mixture or solution comprising a therapeutically effective amount of an active pharmaceutical ingredient together with one or more pharmaceutically acceptable excipients to be administered to a subject (e.g., a human in need thereof).

The term “pharmaceutically acceptable” denotes an attribute of a material which is useful in preparing a pharmaceutical composition that is generally safe, non-toxic, neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic (e.g., the material may be administered to an individual without causing undesirable biological effects or interacting in a deleterious manner with any of the components of the composition in which it is contained).

The terms “pharmaceutically acceptable excipient”, and “pharmaceutically acceptable carrier”, can be used interchangeably and denote any pharmaceutically acceptable ingredient in a pharmaceutical composition having no therapeutic activity and being non-toxic to the subject administered. Non-limiting examples include disintegrators, binders, fillers, solvents, buffers, tonicity agents, stabilizers, antioxidants, surfactants, carriers, diluents, excipients, preservatives or lubricants used in formulating pharmaceutical products.

The term “about” as used herein refers to within +/−10% of the designated amount.

Methods for detection and/or measurement of polypeptides in biological material are well known in the art and include, but are not limited to, Western-blotting, Flow Cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Methods for detection and/or measurement of RNA in biological material are well known in the art and include, but are not limited to, Northern-blotting, RNA protection assay, RT PCR. Suitable methods are described in Molecular Cloning: A Laboratory Manual (Fourth Edition) By Michael R. Green, Joseph Sambrook, Peter MacCallum 2012, 2,028 pp, ISBN 978-1-936113-42-2.

As used herein, the term “antibody” includes but is not limited to a population of immunoglobulin molecules. In some embodiments, the antibodies (e.g., a population of immunoglobulin molecules) can be polyclonal or monoclonal and of any class and isotype, or a fragment of an immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 (human), IgA2 (human), IgAa (canine), IgAb (canine), IgAc (canine), and IgAd (canine). Such fragment generally comprises the portion of the antibody molecule that specifically binds an antigen. For example, a fragment of an immunoglobulin molecule known in the art as Fab, Fab′ or F(ab′)2 is included within the meaning of the term antibody.

The term “label,” as used herein, refers to a detectable compound, composition, or solid support, which can be conjugated directly or indirectly (e.g., via covalent or non-covalent means, alone or encapsulated) to a monoclonal antibody or a protein. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current invention could be, but is not limited to, alkaline phosphatase; glucose-6-phosphate dehydrogenase (“G6PDH”); horseradish peroxidase (HRP); chemiluminescers such as isoluminol, fluorescers such as fluorescein and rhodamine compounds; ribozymes; and dyes. The label may also be a specific binding molecule which itself may be detectable (e.g., biotin, avidin, streptavidin, digioxigenin, maltose, oligohistidine, e.g., hex-histidine (SEQ ID NO: 1), 2, 4-dinitrobenzene, phenylarsenate, ssDNA, dsDNA, and the like). The utilization of a label produces a signal that may be detected by one or more methods (e.g., detection of electromagnetic radiation or direct visualization). In some embodiments, one or more methods (e.g., detection of electromagnetic radiation or direct visualization) may measure the signal.

A monoclonal antibody can be linked to a label using methods well known to those skilled in the art, e.g., Immunochemical Protocols; Methods in Molecular Biology, Vol. 295, edited by R. Burns (2005)).

As used herein, the term “fragment” includes a peptide, polypeptide or protein segment of amino acids of the full-length protein, provided that the fragment retains reactivity with at least one antibody in sera of disease patients.

The term “prognosis” includes a prediction of the probable course and outcome of a disease or the likelihood of recovery from the disease. In some embodiments, the use of statistical algorithms provides a prognosis of the disease in a patient. For example, the prognosis can be surgery, development of a clinical subtype of the disease, development of one or more clinical factors, development of intestinal cancer, or recovery from the disease.

Provided herein are compositions, methods, and compounds for diagnosis, treatment, determining, monitoring, and selecting treatment of cancer, and preferably of AML. In particular aspects, provided herein are compositions and methods for treatment of cancer characterized as CALM-AF10 fusion gene positive or MLL-AF9 fusion gene positive. In some embodiments, the cancer cell is an Acute Myeloid Leukemia (AML) cell, an Acute Lymphoblastic Leukemia (ALL) cell, a multiple myeloma cell, a rhabdomyosarcoma cell, a breast cancer cell, a lymphoma cell, or a skin cancer. In some embodiments, the cancer cell is CALM-AF10 fusion gene positive, MLL-AF9 fusion gene positive, an MLL/KMT2A rearranged AIL cell, an NPM1 mutant AML cell, an AF10-rearranged cell, an AML1-ETO positive cancer cell, or a PML-RARA positive leukemia cell.

In some embodiments, the inhibitory agent comprises a nuclease that catalyzes cleavage of a polynucleotide. Non-limiting examples of nucleases include zinc finger nuclease, fokI nuclease, TALEN nucleases, meganuclease, Cas proteins. In some embodiments, the nuclease is a Cas9 nuclease. In some embodiments, the nuclease is a C2c2 nuclease.

As used herein, Transcription Activator-like Effector Nuclease (TALEN) refers to an enzyme that cuts DNA at a specific sequence recognized by the TAL effector domain. In some embodiments, a TALEN can be used to cleave (e.g., double-strand breaks) at specific target sequences (e.g., in a genome of an organism). In some embodiments, TALEN can be generated by fusing a native or engineered transcription activator-like (TAL) effector, or functional part thereof, to the catalytic domain of an endonuclease, such as, restriction endonuclease Fok1.

In some embodiments, the inhibitory agent comprises a nucleic acid-guided protein complexed with a guide nucleic acid that recognize specific polynucleotide sequences in a cell. In some embodiments, the nucleic acid is a guide RNA. In some embodiments, the inhibitory agent comprises a RNA-guided CRISPR/Cas protein. In some embodiments, the CRISPR/Cas protein is type II CRISPR/Cas protein, a type V CRISPR/Cas protein, a type VII CRISPR/Cas protein, Cas9, CasX, CasY, Cpf1, C2c1, C2c2, or C2c3, or other CRISPR/Cas proteins. In some embodiments, the polynucleotide comprises a RNA sequence that is reverse complementary to a polynucleotide that encodes a SGF29 protein in a cell. In some embodiments, the guide RNA comprises a RNA sequence that is reverse complementary to a polynucleotide that encodes a SGF29 protein in a cell. In a preferred embodiment, the CRISPR/Cas protein is C2c2.

In some embodiments, the inhibitory agent comprises a nucleic acid-guided protein complexed with a guide RNA that recognizes specific polynucleotide sequences in a cell. In some embodiments, the nucleic acid is a guide RNA. In some embodiments, the inhibitory agent comprises a RNA-guided CRISPR/Cas protein. In some embodiments, the CRISPR/Cas protein is type II CRISPR/Cas protein, a type V CRISPR/Cas protein, a type VII CRISPR/Cas protein, Cas9, CasX, CasY, Cpf1, C2c1, C2c2, or C2c3, or other CRISPR/Cas proteins. In some embodiments, the polynucleotide comprises a RNA sequence that is reverse complementary to a DNA that encodes a SGF29 protein in a cell. In some embodiments, the guide RNA comprises a RNA sequence that is reverse complementary to a DNA that encodes a SGF29 protein in a cell. In some embodiments, the CRISPR/Cas protein comprises a mutation in the nuclease domain. In some embodiments, the CRISPR/Cas protein comprises a mutation in the nuclease domain that reduces or abolishes the catalytic activity of the nuclease domain. In some embodiments, the CRISPR/Cas protein comprises a mutation in the nuclease domain that renders the nuclease domain a nickase domain. In some embodiments, the CRISPR/Cas protein is a Cas9 protein comprising mutations D10A and/or H840A compared to the wild type spCas9 protein. In some embodiments, the CRISPR/Cas protein lacks the HNH nuclease domain. In some embodiments, the CRISPR/Cas protein further comprises an effector domain. In some embodiments, the effector domain is a transcriptional repressor domain, DNA methyl transferase domain, histone acetyltransferase domain, histone deacetylase domain, and combinations thereof. A guide nucleic acid (e.g., guide RNA) can bind to a Cas protein and target the Cas protein to a specific location within a target polynucleotide. A guide nucleic acid can comprise a nucleic acid-targeting segment and a Cas protein binding segment.

A guide nucleic acid can refer to a nucleic acid that can hybridize to another nucleic acid, for example, the target polynucleotide in the genome of a cell. A guide nucleic acid can be RNA, for example, a guide RNA. A guide nucleic acid can be DNA. A guide nucleic acid can comprise DNA and RNA. A guide nucleic acid can be single stranded. A guide nucleic acid can be double-stranded. A guide nucleic acid can comprise a nucleotide analog. A guide nucleic acid can comprise a modified nucleotide. The guide nucleic acid can be programmed or designed to bind to a sequence of nucleic acid site-specifically.

A guide nucleic acid can comprise one or more modifications to provide the nucleic acid with a new or enhanced feature. A guide nucleic acid can comprise a nucleic acid affinity tag. A guide nucleic acid can comprise synthetic nucleotide, synthetic nucleotide analog, nucleotide derivatives, and/or modified nucleotides.

The guide nucleic acid can comprise a nucleic acid-targeting region (e.g., a spacer region), for example, at or near the 5′ end or 3′ end, that is complementary to a protospacer sequence in a target polynucleotide. The spacer of a guide nucleic acid can interact with a protospacer in a sequence-specific manner via hybridization (base pairing). The protospacer sequence can be located 5′ or 3′ of protospacer adjacent motif (PAM) in the target polynucleotide. The nucleotide sequence of a spacer region can vary and determines the location within the target nucleic acid with which the guide nucleic acid can interact. The spacer region of a guide nucleic acid can be designed or modified to hybridize to any desired sequence within a target nucleic acid.

A guide nucleic acid can comprise two separate nucleic acid molecules, which can be referred to as a double guide nucleic acid. A guide nucleic acid can comprise a single nucleic acid molecule, which can be referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like and tracrRNA-like molecules.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to target.

In some embodiments, the inhibitory agent comprises a RNA molecule. In some embodiments, the inhibitory agent comprises a non-coding RNA molecule. In some embodiments, the non-coding RNA molecule comprises a microRNA, an siRNA, an anti-sense RNA, or any combination thereof. In some embodiments, the polynucleotide comprises a RNA sequence that is reverse complementary to a DNA that encodes a SGF29 protein in a cell. In some embodiments, the non-coding RNA comprises a siRNA that targets mRNA that encodes a SGF29 protein. In some embodiments, the non-coding RNA comprises a siRNA that targets mRNA that encodes a SGF29 protein. In some embodiments, the non-coding RNA comprises a microRNA that targets mRNA that encodes a SGF29 protein. In some embodiments, the non-coding RNA comprises a microRNA that targets mRNA that encodes a SGF29 protein.

### Therapeutic Approaches

In some embodiments, the compositions described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of a composition or inhibitory agent described herein with one or more other chemical components (e.g. pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

The compositions described herein can be administered to the subject in a variety of ways, including intra-tumorally, parenterally, intramuscularly, colonically, rectally, intraperitoneally, intradermally, subcutaneously, intraperitoneally, or intravenously. In some embodiments, composition describe herein encompasses a small molecule, an antibody, a small interfering RNA (siRNA), short hairpin RNA (shRNA), or an antisense oligonucleotide (ASO). In some embodiments, the small molecule is an inhibitory agent or an inhibitor. In some embodiments, the pharmaceutical compositions can be administered parenterally, intravenously, intramuscularly or orally. The oral agents comprising a small molecule inhibitory agent can be in any suitable form for oral administration, such as liquid, tablets, capsules, or the like. The oral formulations can be further coated or treated to prevent or reduce dissolution in stomach. The compositions of the present invention can be administered to a subject using any suitable methods known in the art. Suitable formulations for use in the present invention and methods of delivery are generally known in the art. For example, the small molecule inhibitory agent described herein can be formulated as pharmaceutical compositions with a pharmaceutically acceptable diluent, carrier or excipient. The compositions may contain pharmaceutically acceptable auxiliary substances as required to approximate physiological conditions. Examples include pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes.

In some embodiments, the pharmaceutical formulation is in the form of a tablet. In other embodiments, pharmaceutical formulations containing a composition or inhibitory agent described herein are in the form of a capsule. In some cases, liquid formulation dosage forms for oral administration are in the form of aqueous suspensions or solutions selected from the group including, but not limited to, aqueous oral dispersions, emulsions, solutions, elixirs, gels, and syrups.

### Biological Samples

In some embodiments, a sample (e.g., a biological sample taken from a subject) may be examined to determine whether, for example, the subject produces an mRNA or a protein subject to regulation by the compositions provided herein. A biological sample can comprise a plurality of biological samples. The plurality of biological samples can contain two or more biological samples. For example, the biological samples may contain from 2, 3, 4, 5, 6, 7, 8, 9, 10, 11, 12, 13, 14, 15, 16, 17, 18, 19, 20, 21, 22, 23, 24, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, 1000, or more biological samples, or from a number of biological samples within a range defined by any of the preceding values. The biological samples can be obtained from a plurality of subjects, giving a plurality of sets of a plurality of samples. The biological samples can be obtained from about 2 to about 1000 subjects, or more. For example, the biological samples may be obtained from at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 68, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, or 1000 or more subjects, or from a number of subjects within a range defined by any of the preceding values.

The biological samples can be obtained from human subjects. The biological samples can be obtained from human subjects at different ages. The human subjects can include male subjects and/or female subjects.

Biological samples can be obtained from any suitable source that allows determination of expression levels of genes (e.g., from cells, tissues, bodily fluids or secretions), or a gene expression product derived therefrom (e.g., nucleic acids, such as DNA or RNA; polypeptides, such as protein or protein fragments). The nature of the biological sample can depend upon the nature of the subject. If a biological sample is from a subject that is a unicellular organism or a multicellular organism with undifferentiated tissue, the biological sample can comprise cells (e.g., a sample of a cell culture, an excision of the organism, or the entire organism). If a biological sample is from a multicellular organism, the biological sample can be a tissue sample, a fluid sample, or a secretion.

The biological samples can be obtained from different tissues. The term tissue is meant to include ensembles of cells that are of a common developmental origin and have similar or identical function. The term tissue is also meant to encompass organs, which can be a functional grouping and organization of cells that can have different origins. The biological sample can be obtained from any tissue.

The biological samples can be obtained from different tissue samples from one or more humans or non-human animals.

The biological samples can be obtained from subjects in different stages of disease progression or different conditions. Different stages of disease progression or different conditions can include healthy, at the onset of primary symptom, at the onset of secondary symptom, at the onset of tertiary symptom, during the course of primary symptom, during the course of secondary symptom, during the course of tertiary symptom, at the end of the primary symptom, at the end of the secondary symptom, at the end of tertiary symptom, after the end of the primary symptom, after the end of the secondary symptom, after the end of the tertiary symptom, or a combination thereof. Different stages of disease progression can be a period of time after being diagnosed or suspected to have a disease. For example, the biological samples may be obtained from at least about, or at least, 1, 2, 3, 4, 5, 6, 7, 8, 9, 10, 11, 12, 13, 14, 15, 16, 17, 18, 19, 20, 21, 22, 23 or 24 hours; 1, 2, 3, 4, 5, 6, 7, 8, 9, 10, 11, 12, 13, 14, 15, 16, 17, 18, 19, 20, 21, 22, 23, 24, 25, 26, 27 or 28 days; 1, 2, 3, 4, 5, 6, 7, 8, 9, 10, 11, 12, 13, 14, 15, 16, 17, 18, 19 or 20 weeks; 1, 2, 3, 4, 5, 6, 7, 8, 9, 10, 11 or 12 months; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 years after being diagnosed or suspected to have a disease. Different stages of disease progression or different conditions can include before, during or after an action or state. For example, disease progression or different conditions may be impacted by treatment with drugs, treatment with a surgery, treatment with a procedure, performance of a standard of care procedure, resting, sleeping, eating, fasting, walking, running, performing a cognitive task, sexual activity, thinking, jumping, urinating, relaxing, being immobilized, being emotionally traumatized, being shock, and the like.

The methods of the present disclosure provide for analysis of a biological sample from a subject or a set of subjects. The subject(s) may be any animal (e.g., a mammal), including but not limited to humans, non-human primates, rodents, dogs, cats, pigs, fish, and the like. The present methods and compositions can apply to biological samples from humans, as described herein.

### Methods of Administering

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Injection can be conducted using sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In some cases, the composition may be sterile. In some cases, the composition may be fluid to the extent that easy syringability exists. In some cases, the composition may be stable under the conditions of manufacture and storage. In some cases, the composition may be preserved against contamination from microorganisms such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. General Methods

Animal Studies: All animal studies were approved and performed as per the guidelines of SBP Medical Discovery Institutional Animal Care and Use Committee. 200 μL of 200,000 UB3 or MOLM13 cells expressing Cas9 and NTC or SGF29 targeting sgRNA were injected intravenously by tail vein injections into NOD/SCID mice (SBP Animal Resources). Mice were monitored every day and sacrificed when critically ill. For MLL-AF10 PDX studies, 150,000 cells expressing split Cas9 and NTC or SGF29 sgRNA were injected into NSG mice (Jackson Laboratories) by tail vein injections. Mice were bled at every 2 to 3 weeks for checking AML cell engraftment by flow cytometry. Animals were sacrificed when critically ill.

Cell culture and reagents: Human leukemia cell lines U937 and MOLM13 (ACC-554, DSMZ), were cultured in RPMI-1640 medium supplemented with 2 mM L-glutamine and sodium pyruvate, 10% Fetal bovine serum and 50 U/mL Penicillin/Streptomycin (Thermo Fisher Scientific, Carlsbad, CA), 2 mM L-glutamine and incubated in 5% CO₂ at 37° C. Murine leukemia cells were cultured in DMEM medium supplemented with 2 mM L-glutamine, 15% FBS and 50 U/mL Penicillin/Streptomycin, in the presence of following cytokines: 10 ng/mL murine interleukin 6 (mIL-6), 6 ng/mL murine interleukin 3 (mIL3) and 20 ng/mL murine stem cell factor (mSCF) (all from Peprotech, Rocky Hill, NJ), and incubated at 5% CO₂ and 37° C. HEK293T cells were cultured in DMEM medium supplemented with 2 mM L-glutamine and sodium pyruvate, 10% FBS and 50 U/mL Penicillin/Streptomycin, and incubated in 5% CO₂ at 37° C. MLL-AF10 patient derived xenograft (PDX) cells were cultured in IMDM medium supplemented with 20% BIT9500 (STEMCELL Technologies), human cytokines and StemRegenin 1 44 (SR1) and UM171.

Data availability: Sequencing data for RNA-seq, ChIP-seq, high-throughput CRISPR screens, and CROPseq are deposited the NCBI GEO under accession number: GSE217829. The sgRNA sequences used for the Human Epigenetic Library is deposited in: https://github.com/kobarbosa/HOX-Manuscript.

Generation of murine reporter leukemia: For mouse experiments, inducible MLL-AF9 murine leukemias were generated in lineage-depleted bone marrow cells of mice with a GFP-Meis1 (12) or CAG-Cas9 (13) background. Hematopoietic stem and progenitor cells (HSPCs) were isolated from bone marrow of C57Bl/6 mice using EasySep lineage depletion (STEMCELL Technologies) kit. HSPCs were cultured overnight in DMEM medium supplemented with 15% FBS and mSCF, mIL6 and mIL3 in a 37° C. incubator with 5% CO₂. The following day, the cells were transduced with viral supernatants containing the MSCV-MLL-AF9-neo and the MSCV-rTTA-2A-BFP constructs by spinfection at 2500 rpm at 30° C. for 90 min. 72 hr post-transduction, BFP+ve cells were sorted using the FACSAria II (BD Biosciences) flow cytometer. In vitro transformed BFP⁺ cells were then injected into sub-lethally irradiated C57Bl/6 recipient mice from the SBP Animal vivarium to generate primary leukemias.

Quantitative RT-PCR: The mRNA levels were measured by real time quantitative PCR (RT-qPCR) using standard protocols. RNA was extracted using the RNA extraction kit (Qiagen, Hilden, Germany) and was used to synthesize cDNA with the Protoscipt® II First Strand cDNA Synthesis Kit (New England Biolabs Inc., Beverly, MA). For PCR-based amplification, cDNA and primers were added to the SYBR Green PCR Master mix (ThermoFisher, Carlsbad, CA). Expression levels for HOXA 7, HOXA9, HOXA10, MEIS1, GAPDH and HPRT were determined with the oligo sequences listed in Table S4 (Barbosa, et al. High Density Domain-Focused CRISPR Screens Reveal Epigenetic Regulators of HOX/MEIS Gene Expression in Acute Myeloid Leukemia. bioRxiv [Internet]. 2022; Available from: doi.org/10.1101/2022.12.12.519332, Supplemental content) and measured on the Stratagene MX3000P (Agilent Technologies). Relative quantity of mRNA was determined by the ΔΔCT method using HPRT as the internal reference.

Colony formation assays: For murine leukemia colony forming cell (CFC) assays, CALM-AF10, MLL-AF9, and MLL-AF10 lines were transduced with retroviral particles encoding 2 sgRNAs targeting Sgf29 exon sequences and one sgRNA targeting an intronic sequence of Sgf29 as a control (sequences provided in Table S4 (Barbosa, et al. High Density Domain-Focused CRISPR Screens Reveal Epigenetic Regulators of HOX/MEIS Gene Expression in Acute Myeloid Leukemia. bioRxiv [Internet]. 2022; Available from: doi.org/10.1101/2022.12.12.519332, Supplemental content)), cloned in pMSCV-U6sgRNA(BbsI)-PGKpuro2ABFP. Cells were transduced by spinfection as described above and cultured for 48 hours in the 37° C. incubator with 5% CO₂. Puromycin (2.5 μg/mL) was then added to the cultures for 48 hours to select for puromycin resistant cells. 1000 cells were plated in 35 mm dish, in duplicates, in 1 mL Methylcellulose-based MethoCult M3234 medium (STEMCELL Technologies) supplemented with 10 ng/mL mIL6, 6 ng/mL mIL3 and 20 ng/mL mSCF (Peprotech) and colonies were scored on day 7. After scoring, colonies were washed with PBS, pooled, and counted for subsequent replating. A sample was taken for cell morphology assessment, performed by spinning 100,000 cells resuspended in 150 μL PBS onto glass slides, using the Cytospin 4 cytocentrifuge (Thermo Scientific) followed by Wright-Giemsa staining. Cells were replated every week for 3 weeks at the same cell number to test secondary and tertiary replating potential. For colony assays with Lin− Sca+c-Kit+ (LSK) murine cells, bone marrow cells from Cas9 transgenic mice (13) were lineage-depleted using the EasySep lineage depletion kit (STEMCELL Technologies) to obtain a lineage negative (Lin−) population. Lin− cells were then stained with the following antibodies: FITC− conjugated Streptavidin (eBioscience), Pacific Blue conjugated Sca-1 (Biolegend, San Diego, CA), and Alexa647-conjugated cKit (Biolegend, San Diego, CA) and sorted for the Lin− Sca-1+ cKit+ (LSK) population using the FACSAria II cytometer (BD Biosciences). Cells were kept in culture overnight and transduced the following day with retroviral particles with sgRNAs targeting Sgf29 and an intron control, as described above. The cells were counted and plated for colony formation in M3434 methylcellulose-based media (STEMCELL Technologies) at a concentration of 10,000 cells per mL, as described above. Colonies were scored on day 10.

Dye-dilution experiments: To trace cell proliferation by flow cytometry, MOLM13 and U937 cells were transduced by spinfection with lentiviral particles containing sgRNAs targeting SGF29 or non-targeting controls (refer to Table S4, Barbosa, et al. High Density Domain-Focused CRISPR Screens Reveal Epigenetic Regulators of HOX/MEIS Gene Expression in Acute Myeloid Leukemia. bioRxiv [Internet]. 2022; Available from: doi.org/10.1101/2022.12.12.519332, Supplemental content), cloned in lentiGuide-Puro vector. Then, cells were incubated overnight and treated with 1 μg/mL puromycin for 3 days. Upon selection, the cells were washed and stained with the CellTrace™ Violet dye (ThermoFisher Scientific), according to the manufacturer's instructions. Cells were maintained in culture in the dark and a sample (>10% of culture volume) was assayed every day for 6 days by flow cytometry.

Viral preparation, transduction and selection: Lentivirus was made from the CROP-seq pooled DNA by transfection of the CROP-seq pool together with Polyethylimine (PEI), pMD2.G, and psPAX2 in HEK293T cells. Viral supernatants were collected at 48- and 72-hours post-transfection, and then filtered (0.45 m) and pooled. Viral supernatants were then concentrated by centrifugation (20,000 rpm, 2 hrs, 4° C.) and used for transduction of UB3 cells by incubation with 0.8 μg/μL of polybrene overnight. Medium was changed after overnight incubation. At 48 h post-transduction, puromycin (1 μg/μL) was added to cell suspensions for 3 days of selection. Cells were pelleted for sequencing 3 days after removal of puromycin.

Single cell sequencing: Two independently transduced samples were sequenced for the CROP-Seq experiment. Cell concentration and viability was assessed using a hemocytometer and viability for all samples was >80%. A total of 18,000 cells per sample were loaded on a Chromium Single Cell Instrument (10× Genomics, Pleasanton, CA). RNAseq libraries were prepared using the Chromium Single Cell 3′ v3.1 Library, Gel Beads & Mutiplex Kit (10× Genomics). CDNA was PCR-amplified and purified using SPRIselect Reagent Kit (Beckman Coulter). Sequencing was performed on the Illumina NovaSeq using the S4 200 kit and PE100 run configuration at the University of California San Diego Center for Epigenomics. Cell Ranger Single-Cell Software Suite (v4.0.0; 10× Genomics) was used for sample demultiplexing, alignment, filtering, and UMI counting.

CROP-seq data analysis: sgRNAs were detected in the fastq files via a custom script, which required a perfect match for either the 10 bp upstream or 10 bp downstream of the sgRNA, as well as the sgRNA sequence with at most 1 mismatch. If a partial sgRNA was located at the end of a sequence but at least 10 bp matched a unique sgRNA (along with the correct neighboring sequence), the read was counted as well (CropSeq_sgRNA_from_fastq.cpp). 10× fastq reads were trimmed using trim-galore (v0.6.6, flags -a AAAAAAAAAAAA (SEQ ID NO: 2) --length 10) which relies on cutadapt (v2.5). Reads were aligned to the genome (hgGRCh38) using STAR (v2.7.3a). All multimapping reads were removed (samtools view -b -q 254), and reads were deduplicated by UMI using custom code (CropSeq_10×_Remove_Duplicates.cpp). Reads were then mapped to genes using bedtools v2.29.2. Reads spanning exon boundaries were split (-split flag used), and all reads were first mapped to exons (flags -f 0.95 -c 7 -o distinct) and then mapped to genes (-f 0.95 -c 4 -o distinct), such that a read mapping to overlapping genes is preferentially assigned to the gene in which it overlaps an exon. Cells with fewer than 10\hat{ }3.5 (3162) UMIs or fewer than 200 expressed genes, or whose barcodes were not on the 10× barcode whitelist were removed. Cells with more than one gRNA were also removed. Finally, genes present in fewer than 3 cells were not included in downstream analyses. General processing and evaluation of sgRNA target gene expression, was performed in R (CROPSeq_HOX.R). All plots in FIG. 24 were generated in R (CROPSeq_HOX.R). UMAP and ridgeplots were generated using the Seurat package. The dendrogram was generated using the R function dist(method=“euclidean”) and hclust(method=“ward.D2”). The heatmap was generated using the pheatmap package.

RNA-sequencing: U937-MEIS1-GFP and MOLM13 cells were plated at a density of 1 million cells per mL in T75 tissue culture flasks and transduced in triplicates with sgRNAs for non-targeting controls or SGF29. Cells were selected with puromycin as described above. 2 million cells were harvested at 7 days post-selection from every replicate and RNA was extracted using the RNeasy Mini kit (Qiagen, Hilden, Germany). RNA-seq Libraries were prepared using NEBNext Ultra II Directional RNA Library Prep Kit (New England Biolabs, Ipswich, MA) for Illumina as per the manufacturer's protocol. For each sample, 900 ng of total RNA was amplified in 10 cycles of PCR amplification. Sequencing was performed to obtain 1×75 bp reads on the Illumina NextSeq500 by the Genomics core at SBP Medical Discovery Institute. Next-generation sequencing data were demultiplexed and processed with the Illumina Basespace RNA-Seq Differential Expression Analysis workflow.

RNA-sequencing data analysis: Raw reads were preprocessed by trimming Illumina Truseq adapters, polyA, and polyT sequences using cutadapt v2.3226 with parameters “cutadapt -j 4 -m 20 --interleaved - aAGATCGGAAGAGCACACGTCTGAACTCCAGTCAC (SEQ ID NO: 3) - AAGATCGGAAGAGCGTCGTGTAGGGAAAGAGTGT (SEQ ID NO: 4) Fastq1 Fastq2|cutadapt --interleaved -j 4 -m 20 -a “A{100} (SEQ ID NO: 5)”-A “A{100} (SEQ ID NO: 5)”-|cutadapt -j 4 -m 20 -a “T{100} (SEQ ID NO: 6)”-A “T{100} (SEQ ID NO: 6)”-”. Trimmed reads were subsequently aligned to human genome version hg38 using STAR aligner v2.7.0d_0221 227 with parameters according to ENCODE long RNA-seq pipeline (https://github.com/ENCODEDCC/long-rna-seq-pipeline). Gene expression levels were quantified using RSEM v1.3.1 228. Ensembl v84 gene annotations were used for the alignment and quantification steps. RNA-seq sequence, alignment, and quantification qualities were assessed using FastQC v0.11.5 (https://www.bioinformatics.babraham.ac.uk/projects/fastqc/) and MultiQC v1.8 229. Lowly expressed genes were filtered out by retaining genes with estimated counts (from RSEM)≥number of samples times 5. Filtered estimated read counts from RSEM were used for differential expression comparisons using the Wald test implemented in the R Bioconductor package DESeq2 v1.22.2 based on generalized linear model and negative binomial distribution (Love, Huber et al. 2014). Genes with Benjamini-Hochberg corrected p-value <0.05 and fold change ≥2.0 or ≤2.0 were selected as differentially expressed genes. Pathway analyses of differential expression comparisons were performed using Ingenuity Pathway Analysis (Qiagen, Redwood City, USA).

Chromatin immunoprecipitation and sequencing (ChIP-Seq): UB3 cells expressing Cas9 were transduced with sgRNAs for a non-targeting control or SGF29, as described for the dye-dilution experiment. Chromatin immunoprecipitation was performed. Cells were fixed with 1% formaldehyde and chromatin was sheared using a Bioruptor™ (Diagenode Inc, NJ) in 15 cycles (each one in high-setting for 30 s on and 30 s off), at 4° C. Chromatin was immunoprecipitated using antibodies for normal rabbit IgG (Cell Signaling Technology, Cat. No, 2729) and Histone H3K9ac (Abcam, ab4441). For SGF29 binding studies, U937 cells transduced with 3× Flag tagged SGF29 wt or D196R mutant viral supernatants were fixed with 1% formaldehyde for 15 minutes and chromatin was sheared as described earlier. Chromatin was immunoprecipitated using Anti-FLAG M2 affinity beads (Sigma Aldrich, #A2220). Library preparation on eluted DNA was performed using the NEBNext Ultra II DNA library prep kit for Illumina (E7645S and E7600S) as per the manufacturer's protocol. DNA libraries were sequenced in paired-end 150 bp reads in a NovaSeq600 sequencer (Illumina, San Diego, CA) by Novogene Corporation (Sacramento, CA).

Chromatin enrichment for proteomics (ChEP): Five million U937 cells were transduced in 4 replicates with concentrated lentiviral supernatants containing a non-targeting control or sgRNAs targeting CSNK2A1 or SGF29 in the pKLV2-U6gRNA5(BbsI)-PGKpuro-2ABFP backbone (see Table 2 for sequences). 48 hours after transduction, cells were treated with 1 μg/mL puromycin for 3 days. The cultures were grown for 7 days and counted for chromatin enrichment for proteomics procedure. Briefly, 100 million cells per replicate were collected by centrifugation for 5 minutes at 600×g at room temperature and washed with pre-warmed PBS. Cells were then resuspended in 1% formaldehyde in pre-warmed PBS and incubated for 10 min at 37° C. in a rotator. Crosslinking was halted by adding glycine to a final concentration of 0.25M and incubating for 5 min at room temperature in a rotator. Cells were then washed with pre-warmed PBS and pellets were collected by centrifugation at room temperature for 5 min at 600×g and stored at −80° C. Pellets were thawed and resuspended in 1 mL cold cell lysis buffer (25 mM Tris, 0.1% Triton X-100, 85 mM KCl, and protease inhibitors), transferred to 2 mL tubes, and homogenized carefully with a 200 μL pipette tip. The samples were then centrifuged for 2300×g for 5 min at 4° C. and the supernatants containing the cytoplasmic fraction were recovered and stored at −80°. The nuclei pellets were resuspended in 500 μL cell lysis buffer containing 200 μl/mL RNAse A and were incubated for 15 min at 37° C. Samples were then kept on ice and centrifuged at 2,300×g for 10 min at 4° C. The supernatants were then discarded, and pellets were resuspended in 500 μL SDS buffer (50 mM Tris, 10 mM EDTA, 4% SDS, and protease inhibitors) with 200 μL pipette tips and incubated for 10 min at room temperature. Samples were then centrifuged at 16,000×g for 30 min at room temperature and the supernatants were discarded. The pellets were washed by resuspending in 500 μL SDS buffer with a 200 μL tip, then adding 1.5 mL urea buffer (10 mM Tris, 1 mM EDTA, and 8 M urea) and mixing by inversion several times. The tubes were then spun down at 16,000×g for 25 min at room temperature and supernatants were discarded. Urea was washed out by resuspending in 500 μL SDS buffer with a 200 μL tip, then adding 1.5 mL SDS buffer and mixing by inversion several times. The tubes were spun down at 16,000×g for 25 min at room temperature and the pellets were covered with storage buffer (10 mM Tris, 1 mM EDTA, 25 mM NaCl, 10% glycerol, and protease inhibitors) and flicked to dislodge the pellets. The pellets were transferred to Bioruptor® shearing tubes (Diagenode Inc, Denville, NJ) and sonicated in a Bioruptor® (Diagenode Inc., Denville, NJ) for 15 min in 15 cycles (each 30 s on, 30 s off on high intensity setting). The samples were centrifuged at 16,000×g for 30 min at 4° C. and the supernatants were transferred to a new tube and stored at −80° C. for mass spectrometry analysis.

TABLE 2

| | Gene and | |
|---|---|---|
| Organism | sgRNA No. | sgRNA sequence |
| Human | AFF2-1 | CTTGTCCGCTACGTTCGCCA |
| | | (SEQ ID NO: 7) |
| Human | AFF2-2 | GAGCTTCATGACCCACCAAG |
| | | (SEQ ID NO: 8) |
| Human | AFF2-3 | GTACTCTAGAGAGTAAGTCA |
| | | (SEQ ID NO: 9) |
| Human | CCDC101-1 | TATCTCTGAGCCACATTGAG |
| | | (SEQ ID NO: 10) |
| Human | CCDC101-3 | CAATGTGGCTCAGAGATACG |
| | | (SEQ ID NO: 11) |
| Human | CSNK2A1-1 | TGAGGATAGCCAAGGTTCTG |
| | | (SEQ ID NO: 12) |
| Human | CSNK2A1-2 | AGTCACATGTGGTGGAATGG |
| | | (SEQ ID NO: 13) |
| Human | CSNK2A1-3 | GATTGATCATGAGCACAGAA |
| | | (SEQ ID NO: 14) |
| Human | CSNK2B-2 | CAGGCAGCCGAGATGCTTTA |
| | | (SEQ ID NO: 15) |
| Human | CSNK2B-3 | TCTCGGCTGCCTGCTCAATC |
| | | (SEQ ID NO: 16) |
| Human | DOT1L-1 | GGGAGCGAATCGCCAACACG |
| | | (SEQ ID NO: 17) |
| Human | DOT1L-2 | GCAGCAGGTCTACAACCACT |
| | | (SEQ ID NO: 18) |
| Human | DOT1L-3 | GTCCACAAACAGGTCGTCGT |
| | | (SEQ ID NO: 19) |
| Human | ENY2-1 | TAGCTCTCAGCAACTCTTTG |
| | | (SEQ ID NO: 20) |
| Human | ENY2-2 | CAGTTGAAGGCACACTGTAA |
| | | (SEQ ID NO: 21) |
| Human | ENY2-3 | AAAGGCTGGCATGCTGAGCA |
| | | (SEQ ID NO: 22) |
| Human | JADE3-1 | TTTGGACATAACACACATTG |
| | | (SEQ ID NO: 23) |
| Human | JADE3-2 | AGCCCATTTAGTCCCTGTCT |
| | | (SEQ ID NO: 24) |
| Human | JADE3-3 | GTCACCTGTGCCTTTGAGCA |
| | | (SEQ ID NO: 25) |
| Human | KAT7-1 | TCTCATCGTGAGATACATTG |
| | | (SEQ ID NO: 26) |
| Human | KAT7-2 | TGTCCTAGAGAGTTACAGCC |
| | | (SEQ ID NO: 27) |
| Human | KAT7-3 | ACAGACAGTTCAGAAAGTGA |
| | | (SEQ ID NO: 28) |
| Human | KMT2A-1 | GATCTGAAAGCCATCATCAC |
| | | (SEQ ID NO: 29) |
| Human | KMT2A-2 | TCATAACATTTGTCACAGAG |
| | | (SEQ ID NO: 30) |
| Human | KMT2A-3 | CCATCGGCACCAACCTGCGC |
| | | (SEQ ID NO: 31) |
| Human | MLLT1-1 | GAGCTTGTACCGGAACTCCG |
| | | (SEQ ID NO: 32) |
| Human | MLLT1-3 | CAGCCATGGACAATCAGGTG |
| | | (SEQ ID NO: 33) |
| Human | NTC-1 | ATGGAAGAGCGTCATGACTT |
| | | (SEQ ID NO: 34) |
| Human | NTC-2 | CCAGTTGCTCTGGGGGAACA |
| | | (SEQ ID NO: 35) |
| Human | BRD4-1 | CACCAAACTCCTGAGCATCA |
| | | (SEQ ID NO: 36) |
| Human | BRD4-2 | CTGAGCATCACGGTACTCAC |
| | | (SEQ ID NO: 37) |
| Human | MLLT10-1 | ACATGTCATGCAAGCACCAG |
| | | (SEQ ID NO: 38) |
| Human | MLLT10-2 | TCCATCATGCCGATACTGCA |
| | | (SEQ ID NO: 39) |
| Human | MLLT3-1 | CAGCGGAGGTGATTCACTGG |
| | | (SEQ ID NO: 40) |
| Human | MLLT6-1 | GCTGTGCCCACACAAAGACG |
| | | (SEQ ID NO: 41) |
| Human | MLLT6-2 | CGTTGGCAAATTGCACCTCG |
| | | (SEQ ID NO: 42) |
| Human | MLLT3-1 | TGCAGGTGAAGCTGGAGCTG |
| | | (SEQ ID NO: 43) |
| Human | AFF4-1 | GACTTCCAGTAGCTCCAAGG |
| | | (SEQ ID NO: 44) |
| Human | AFF4-2 | ATGTGCTGCGTATGAAAGAA |
| | | (SEQ ID NO: 45) |
| Mouse | Sgf29-1 | AACCGCCAAGACTGATGCGG |
| | | (SEQ ID NO: 46) |
| Mouse | Sgf29-2 | CACAACCGCCAAGACTGATG |
| | | (SEQ ID NO: 47) |
| Mouse | Sgf29-3 | AGAGGCCTCGAAGCTTTGTC |
| | | (SEQ ID NO: 48) |
| Mouse | Sgf29-4 | GTCGAGAAGTGAGCACAACT |
| | | (SEQ ID NO: 49) |
| Mouse | Sgf29- | CCCTCTGGTCCTGCTGTGTC |
| | intron-1 | (SEQ ID NO: 50) |
| Mouse | Sgf29- | ACTGTAATCATCTCAAAATT |
| | intron-2 | (SEQ ID NO: 51) |

LC-MS/MS analysis: For the chromatin-enriched for proteomics samples, 30 μg of each sample were processed using the ProTiFi S-trap™ digestion system. Mass spectrometry data were acquired on an Orbitrap Exploris 480 (ThermoFisher, Bremen, Germany) instrument at the Cedars Sinai Proteomics and Metabolomics Core in the Advanced Clinical Biosystems Research Institute. Desalted peptides were separated on an Ultimate 3000 ultra-high-pressure chromatography system with a 60-min gradient. Peptides were separated on a gradient of 1% B organic phase for 2 minutes, 1-5% B for 0.5 minutes, 5-9% B for 3.5 minutes, 9-27% B for 39 minutes, and 27-44% B for the final 15 minutes on a C18 column (15 cm length, 300 m diameter) at a flow rate of 9.5 nL/min. Source parameters included spray voltage at 3 kV, capillary temp of 300° C., and RF funnel level of 40%. MS1 resolutions were set to 60,000 and AGC was set to “standard” with ion transmission of 100 ms. Mass range of 400-1000 and AGC target value for fragment spectra of 300% were used. Peptide ions were fragmented at a normalized collision energy of 30%. Fragmented ions were detected across 50 DIA windows of 12 Da. MS2 resolutions were set to 15,000 with an ion transmission time of 25 ms. All data were acquired in profile mode using positive polarity. The data were searched using the DIA-NN tool against a human in silico digested sequence reference library.

Data analysis for ChEP: Protein peak data (mz/rt) were analyzed using MetaboAnalyst 5.0. Missing values were imputed by replacing them with ⅕^{th} of the minimum positive values of their corresponding variables. No data filtering was applied. Important features were selected based on fold change analysis with a threshold of 2 and t-test P. values below 0.05.

Western and dot blotting: Whole cells were lysed in cold RIPA buffer containing the Halt™ protease inhibitor cocktail for 30 min on ice (ThermoFisher Scientific, Carlsbad, CA). Protein supernatant was collected after 30 min of 12,000 rpm centrifugation at 4° C. and stored at −80° C. until use. Protein concentrations from whole cell lysates were determined using the Pierce™ BCA protein assay kit (ThermoFisher Scientific). For the immunoblotting, the following antibodies were used: KAT2A (Abcam, ab217876), Flag M2 (Sigma-Aldrich, F1804), Vinculin (Sigma-Aldrich, V9131), H3 (Abcam, ab1791), and SGF29 (Abcam, ab204367). Protein samples (50 g total protein) were resolved by SDS-PAGE using 4-12% Bis-Tris Bolt™ gels (Invitrogen, NW04120BOX) and transferred to iBlot2™ nitrocellulose membranes (ThermoFisher Scientific, IB23002). The membranes were blocked with 5% non-fat milk in Tris-buffered Saline containing 0.01% tween-20 (0.01% TBST). The membranes were probed with species-specific goat anti-rabbit (MilliporeSigma, AP307P) or goat anti-mouse (ThermoFisher Scientific, 31446) HRP-conjugated secondary antibody and detected with enhanced chemiluminescence (ECL) detection kit (Thermo Fisher, Carlsbad, CA).

Cell Cycle Analysis: 500,000 MLL-AF10 PDX cells expressing NTC or SGf29 sgRNA were washed with cold PBS and fixed with chilled 70% ethanol. Fixed cells were incubated at −20° C. for 1 hr. Cells were then centrifuged at 2000 rpm for 3 min. at 4° C. Pellets were resuspended in PBS containing RNAse A and Propidium Iodide (PI) and incubated at 37° C. for 30 min. in dark and analysed using flow cytometry.

### Example 2. Epigenetic CRISPR Screen

The pooled epigenetics CRISPR library virus was produced using HEK293T cells transfected with Polyethylimine (PEI), pMD2.G, and psPAX2. 30 million UB3 cells were transduced with the pooled epigenetics library lentivirus in RPMI medium supplemented with 10% fetal bovine serum, antibiotics, and 8 μg/mL polybrene. The medium was changed 24 h after transduction to remove polybrene and cells were plated in fresh culture medium. 48 h after transduction, puromycin was added at a concentration of 1 μg/mL to select for cells transduced with the sgRNA library and then removed after 72 h. The virus titer was measured by infection of cells with serially diluted virus. To ensure transduction of a single sgRNA per cell, the multiplicity of infection (MOI) was set to 0.3˜0.4. Adequate representation of sgRNAs during the screen was ensured by keeping >1000× cells in culture relative to the library size. 10 million cells per replicate were harvested 3 days after puromycin removal for an initial time point (TO). Cells were FACS-sorted 5 days later to collect the upper and lower quartiles on the basis of GFP-expression using the FACSAria II Instrument (BD Biosciences) at the Sanford Burnham Prebys Flow Cytometry Core. Genomic DNA extraction from the TO and GFP-low and -high cells was performed using the Zymo QuickDNA™ Midiprep Plus Kit (Zymo Research, Cat. D4068), according to the manufacturer's instructions. A two-step PCR-amplification for sequencing library preparation was conducted with TaKaRa Ex Taq™ Polymerase (TaKara, Cat. RR001) and custom primers to achieve adequate sequencing coverage in 1×75 bp single-end reads, following published guidelines. Barcoded libraries were pooled and sequenced using an Illumina Hiseq 500.

### Example 3. CRISPR Competition Experiments

To assess the candidate hits in independent assays, two top ranked individual sgRNAs from the sgRNA library were cloned for CSNK2A1, JADE3, AFF2, CCDC101, CSNK2B, DOT1L, ENY2, KAT7, KMT2A, MLLT1, and for non-targeting controls in the pKLV2-U6gRNA5(BbsI)-PGKpuro2ABFP-W vector 218 (sgRNA sequences provided in Table 2). Lentiviral supernatants were made from these constructs in a 96-well arrayed format. 10,000 UB3 cells were plated in a retronectin (Takara, #T202) coated flat bottom 96-well plate and transduced with the sgRNA viral supernatants by spinfection in polybrene-supplemented medium at an MOI of ˜0.5. Cells were maintained in culture in 96-well plate format and assayed for proliferation every 3 days. A sample >10% of the culture volume was stained with SytoxRed (1:1000) (Thermo Fisher, #S34859) in PBS and monitored for the percentage of blue fluorescent protein (BFP) expressing cells progressively up to 21 days using high-throughput flow-cytometry.

### Example 4. Competition Assays Reveal the Role of MEIS1 Regulators in AML Cell Growth

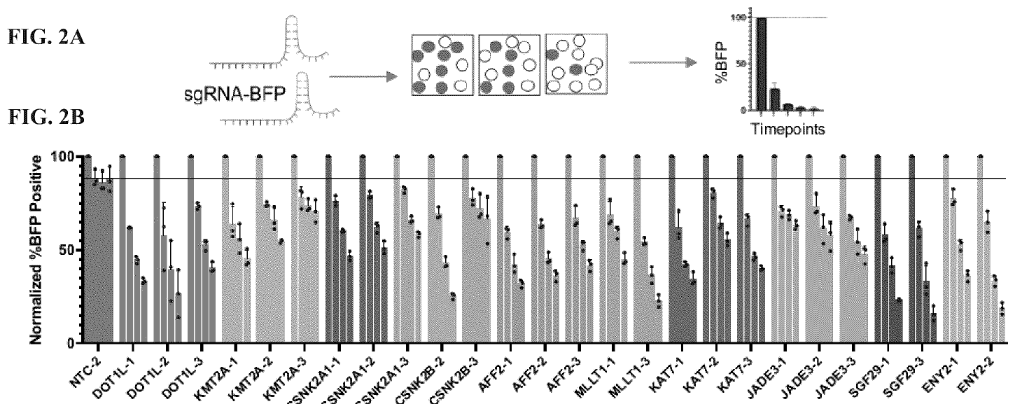

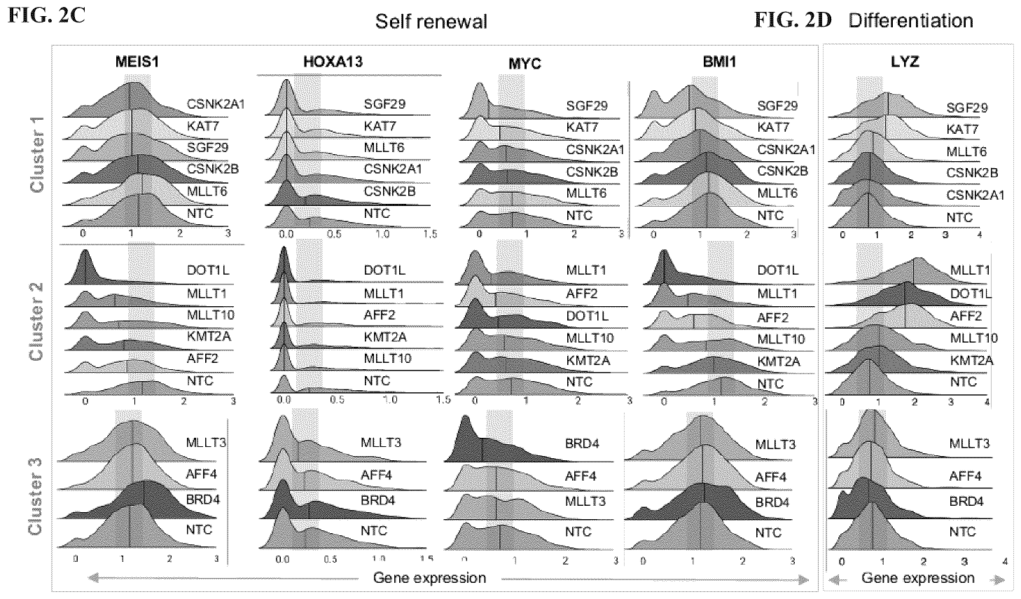

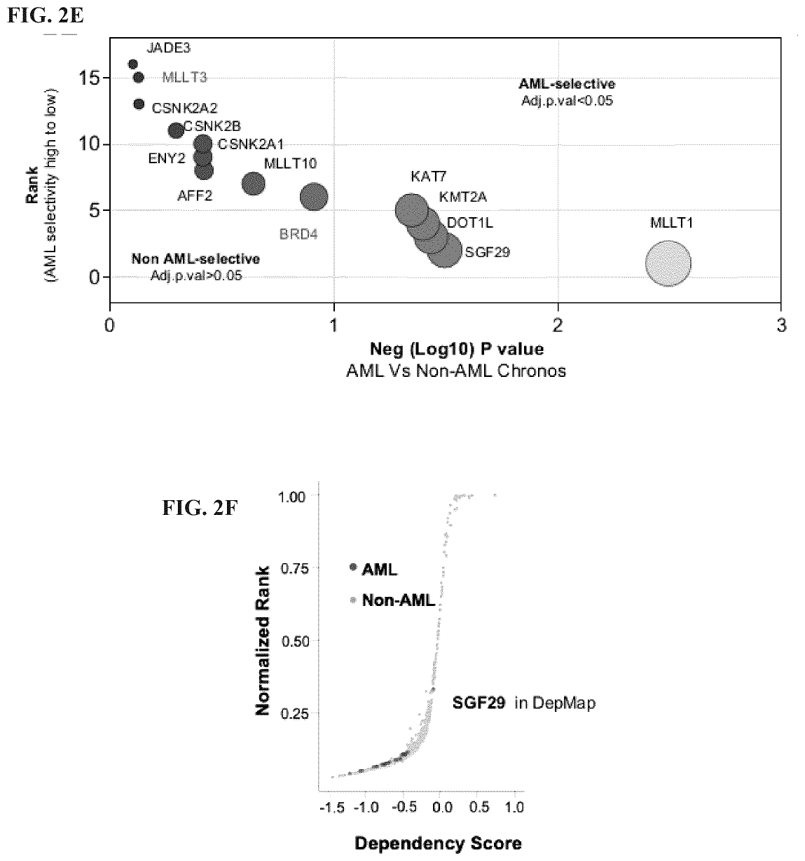

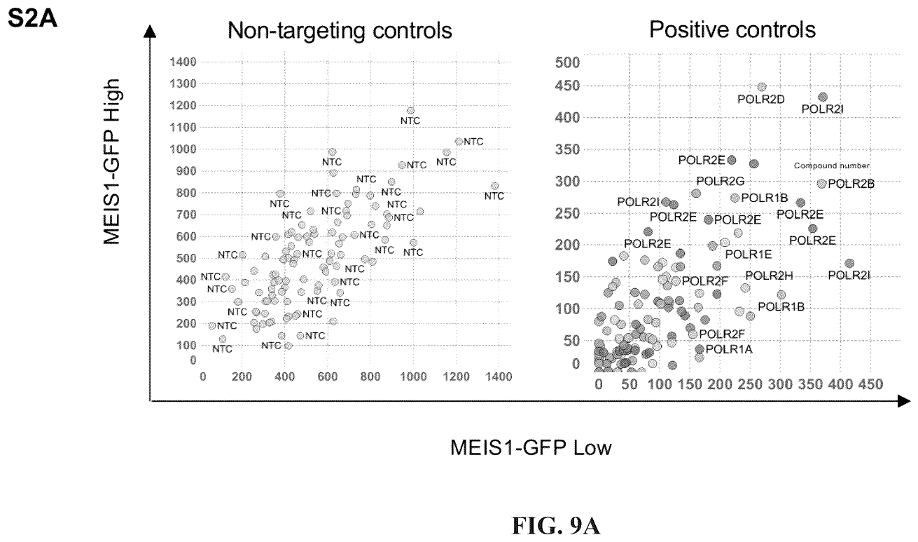

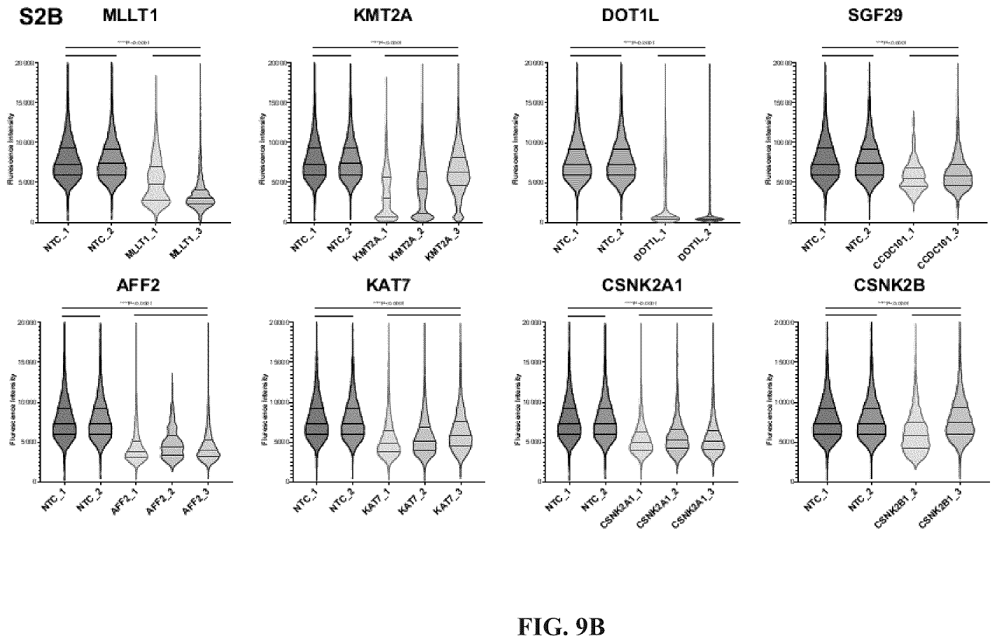

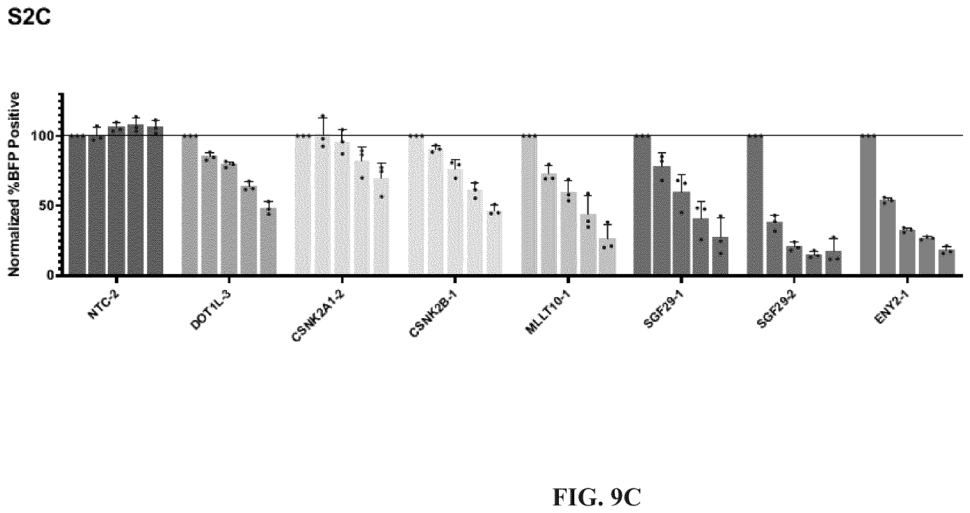

The requirement of top candidate MEIS1 regulators for U937 cell growth was tested using arrayed CRISPR competition assays in UB3 cells (see FIG. 2A for schematic). Specifically, 2-3 sgRNAs were cloned for each of the top candidate hits (SGF29, ENY2, AFF2, CSNK2A1, CSNK2B, MLLT1, KAT7, and JADE3), in addition to DOT1L as a positive control, and non-targeting controls (NTC). For this, a plasmid vector co-expressing a blue fluorescence protein (BFP) and lentivirally transduced U937 cells at a ˜50% transduction rate was used. The populations were sampled by flow cytometry for up to 14 days and observed a significant (P<0.05, n=3) and progressive decline in the percentage of BFP positive cells targeted by all the candidate hits compared to NTCs over time (FIG. 2B). In addition, a significant drop in eGFP fluorescence (P<0.05, n=3) was observed, for multiple sgRNAs targeting candidate hits, confirming a strong reduction in MEIS1 expression (FIG. 9B). In sum, these results indicate phenotypic screening strategy uncovered high-confidence MEIS1 regulators that affect the growth of U937 cells. In order to rule out that this antiproliferative effect was limited to the CALM-AF10 positive cell line U937, some of the novel MEIS1 regulators, including CSNK2A1, ENY2, and SGF29 and controls in a Cas9-expressing MLL-AF9 fusion positive MOLM13 cell line (FIG. 9C) were tested. CRISPR deletion of these genes also led to a progressive decline in the percentage of BFP positive cells compared to NTC-transduced cells, indicating that growth of MOLM13 cells also depended on these genes (FIG. 9C). Surprisingly, among these genes, the deletion of SGF29 and ENY2 had the most detrimental effects on the growth of both U937 as well as MOLM13-cells (FIG. 2B and FIG. 9C).

### Example 5. CRISPR Droplet-Sequencing Reveals Leukemia Oncotranscriptome Targets of Candidate Regulators

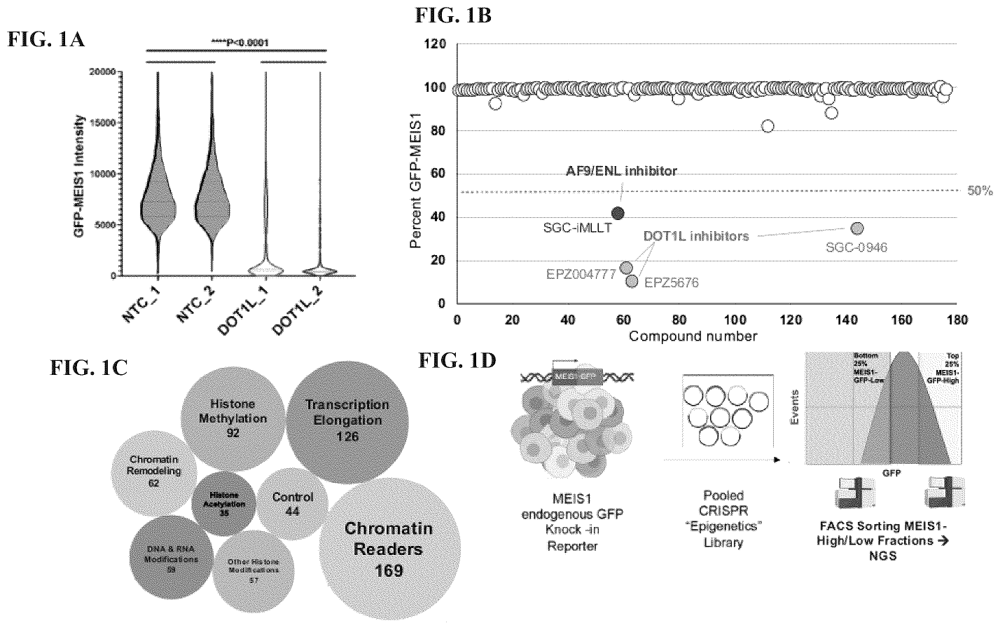

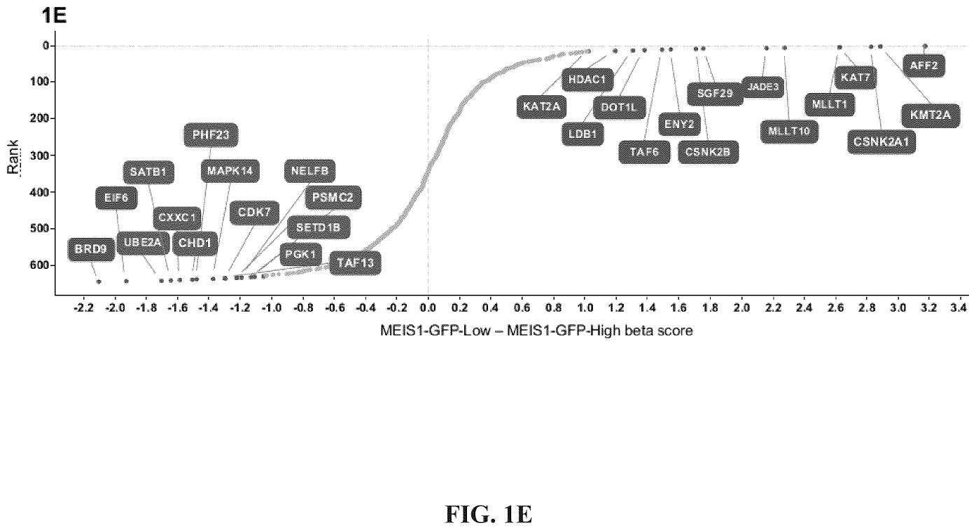

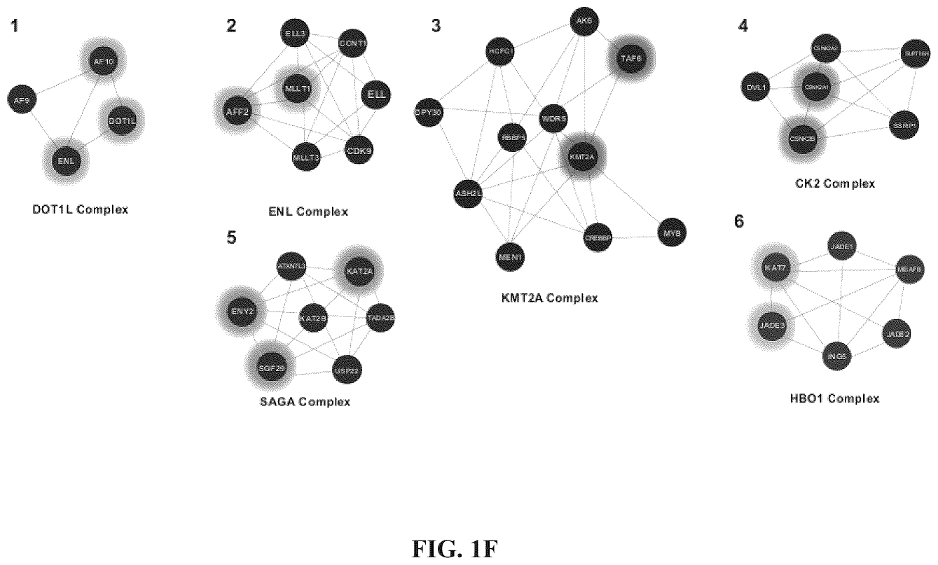

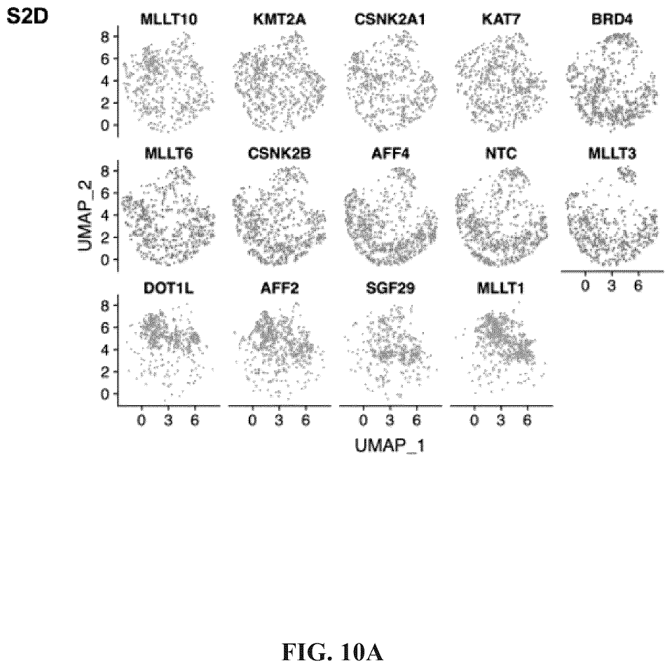

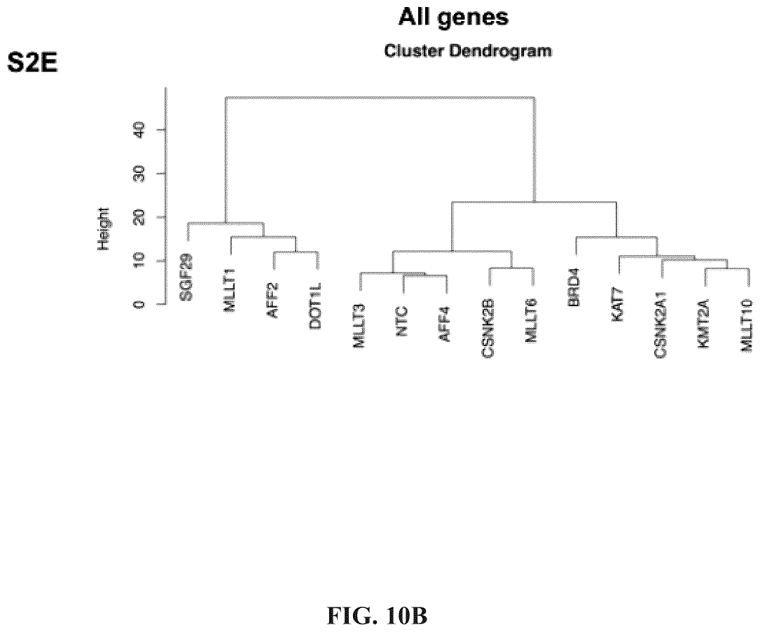

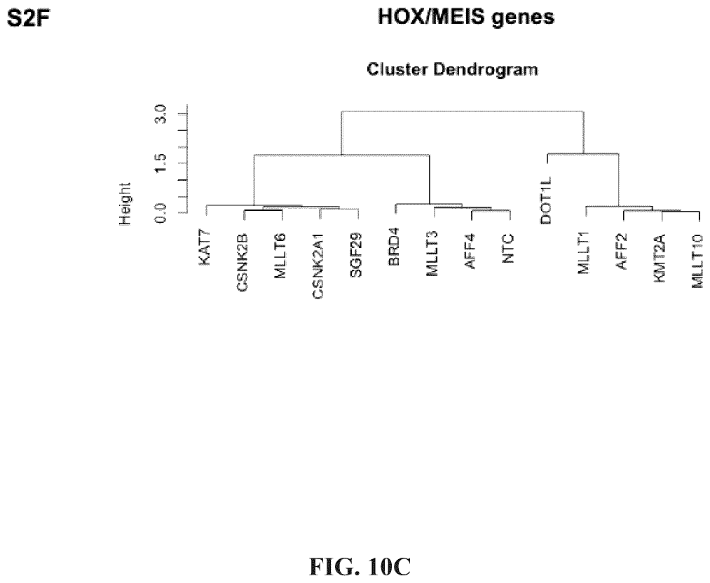

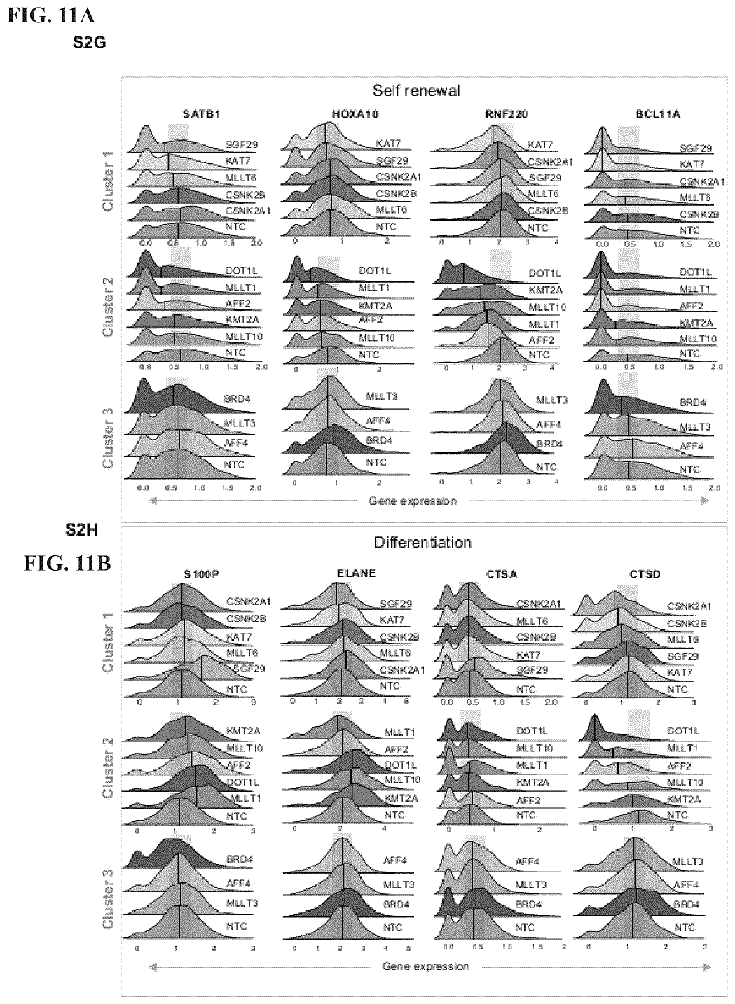

MEIS1 was used as a surrogate reporter for the screen, steps were then taken to further evaluate the following: a) whether candidate hits could regulate the expression not only of MEIS1, but also of other AML-associated oncogenes and b) which epigenetic regulators from our hits had the most pronounced effects on sustaining transcription of AML-promoting oncogenes. For this, targeted, a small pooled CRISPR library containing 2-3 validated sgRNAs were generated per candidate gene together with non-targeting controls (a total of 29 sgRNAs) and performed a single cell RNA-sequencing experiment in the UB3 cell line. Specifically, CRISPR droplet sequencing (CROP-Seq) was used, which allows for matching the single-cell RNA-sequencing profile of each cell to the sgRNA expressed within it, and thus infer knockout signatures of each of the CRISPR-deleted genes. After the assignment of sgRNAs to the pool of U937 cells, unbiased clustering of whole transcriptome single cell RNA-seq data was performed using the Ward. D2 minimum variance method (see Supplemental Methods) revealing the relatedness of each of the perturbations (FIGS. 9C and 9E). While the transcriptomes of CSNK2A1-, KMT2A- and MLLT10-knockout clustered together, DOT1L-, MLLT1-, AFF2-and SGF29-deleted transcriptomes formed a different cluster, indicating transcriptional similarities. Lastly, sgRNAs for control genes were included which are either chromatin readers that do not show HOX/MEIS-specific gene regulatory activity in U937 cells (MLLT3, MLLT6), or have non-selective transcriptional effects (BRD4). Surprisingly, analysis of the CROP-Seq data revealed that CRISPR deletion of several of the candidate epigenetic regulators, including AFF2, MLLT1, MLLT10, DOT1L, KAT7, SGF29, CSNK2A, and CSNK2B led to the downregulation not only of MEIS1, but also of several other leukemia and LSC-associated oncogenes (FIG. 2C and FIGS. 11A and 10C, and Table S3). These downregulated genes included key genes of the HOXA cluster, BMI1, SATB1, RNF220, and MSI2. Moreover, their deletion also led to a concomitant increase in the expression of differentiation-associated genes such as lysozyme (LYZ), ELANE, the cathepsins CTSA and CTSD, and the S100A8/A9 proteins that are typically highly expressed in differentiated myeloid cells or involved in myeloid differentiation (FIG. 2D and FIG. 11B). We then performed clustering of the single cell RNA-seq data using to investigate the transcriptomic relatedness of each perturbation to the others in terms of their effects on HOXA/MEIS expression (see Supplemental Methods). Analysis identified 3 major clusters. The first cluster comprises DOT1L, MLLT1, MLLT10, and AFF2. This cluster possibly reflects overlapping activities of these proteins that have been described to interact in multi-protein complexes (23-25). Interestingly, AFF2, but not the super-elongation (SEC) complex constituent AFF4 was identified as a regulator of HOX/MEIS1 expression in U937 cells (FIG. 1E, FIG. 10C and Table S3). Similarly, the deletion of MLLT1, but not its homolog MLLT3, modulated HOX/MEIS expression in the UB3 cells, consistent with a prior study in a KMT2A-rearranged AML. Cluster 2 consisted of the genes KAT7, CSNK2A1, CSNK2B, and SGF29, whose deletion also had potent effects on the downregulation of MEIS1 and other leukemia oncogenes (FIG. 2C, FIG. 10C and Table S3). Cluster 3 consisted of the control genes included in the CROP-Seq study, namely AFF4 and the chromatin readers BRD4 and MLLT3, whose deletion had no significant effects on HOX/MEIS or on differentiation-associated signatures in the setting of the UB3 cell line. Taken together, these studies showed that several epigenetic regulators identified in the eGFP-MEIS1 screen were important for sustaining key LSC signature genes and in reversing the differentiation block in U937 cells.

### Example 6. CRISPR Dependency Assays Earmark SGF29 as an AML-Selective Dependency

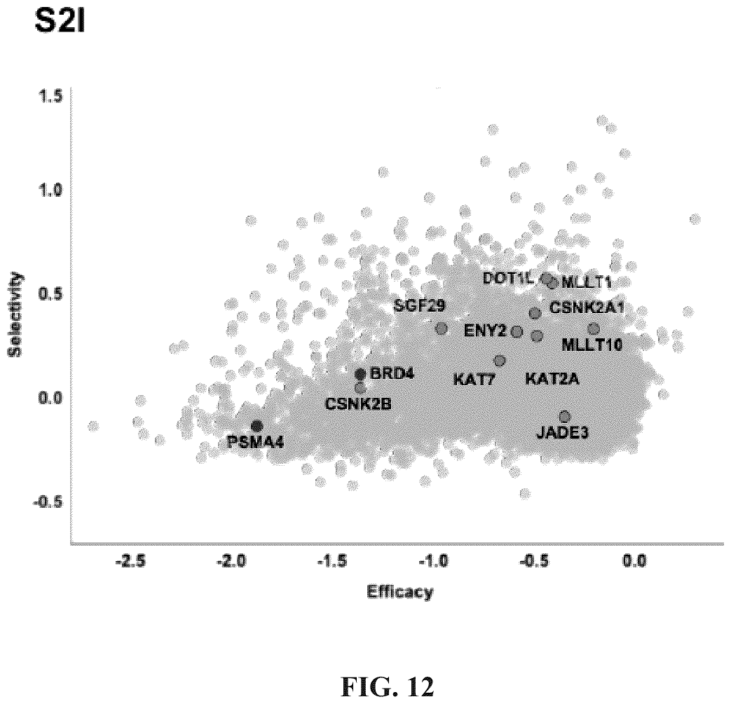

A challenge in developing epigenetic candidates as therapeutic candidates is that drugging some of these candidates has the potential for non-selective toxicity. A way of assessing the selectivity of a candidate gene as a therapeutic target is to investigate the effect of its genetic depletion in the cancer dependency maps (DepMap) dataset, a database of genome-scale functional genetic screens in hundreds of cancer cell lines. Top candidate epigenetic regulators were assessed for efficacy (the degree to which CRISPR knockout of the candidate gene reduces cell fitness in sensitive lines), and selectivity (the degree of differences in its essentiality across all cancer cell lines) using the Shiny DepMap application which combines CRISPR and shRNA data from diverse cancer cell lines. Analyses showed that some of the novel candidate hits identified in the screen, including SGF29, ENY2 and CSNK2A1, as well as previously characterized genes such as DOT1L, MLLT1, MLLT10 and KAT7 had a higher selectivity compared to pan-essential genes such as PSMA4 or even the chromatin reader BRD4 (FIG. 12).

Next, since several candidate epigenetic regulators seemed to regulate the expression of AML-promoting oncogenes, the differences in median CRISPR knockout fitness scores (Chronos) between 26 AML cell lines in the AML DepMap data and 1,060 non-AML cancer cell lines were assessed. This analysis showed that while some of the hits identified in the screen were highly AML selective, others were not. Specifically, the most AML-selective genes from candidate hits were MLLT1, SGF29, DOT1L, AFF2 and KMT2A (ranked in decreasing order of AML selectivity as assessed by Wilcox test of AIL Vs Non-AML cell line dependency scores) (FIG. 2D). Other chromatin regulators, namely MLLT10, CSNK2A1/2, CSNK2B, JADE3 and ENY2 were not AML selective (FDR p value <0.05). Depletion of the AML-selective hits significantly impaired the fitness of AML compared to non-AML cell lines. This data provides an indication that the AML-selective candidate genes may serve as more selective therapeutic targets in terms of designing anti-AML therapies. Of particular interest was SGF29 effect on leukemogenic gene expression programs and high AML selectivity across 1,086 cancer cell types (FIGS. 2E and 2F).

### Example 7. SGF29 Deletion has Pronounced Anti-Leukemia Effects in Cell-Line-Derived and Patient-Derived AML Models

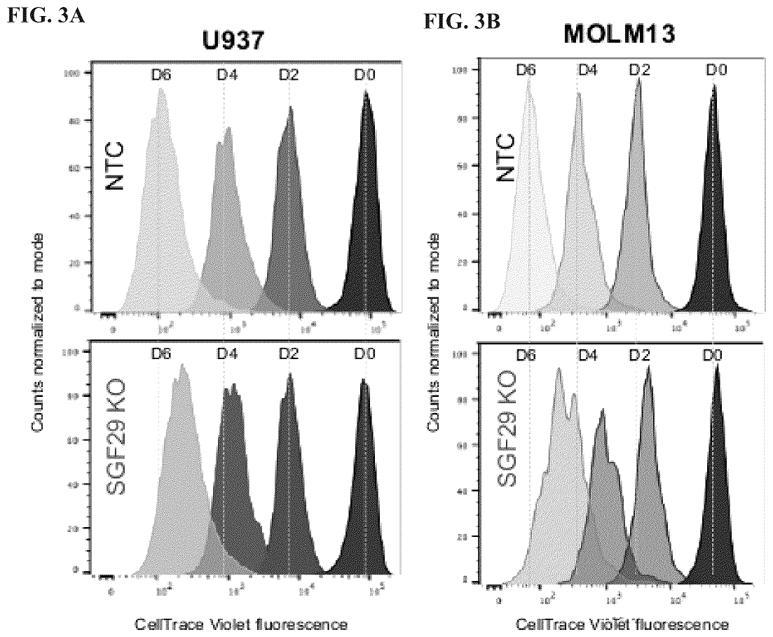

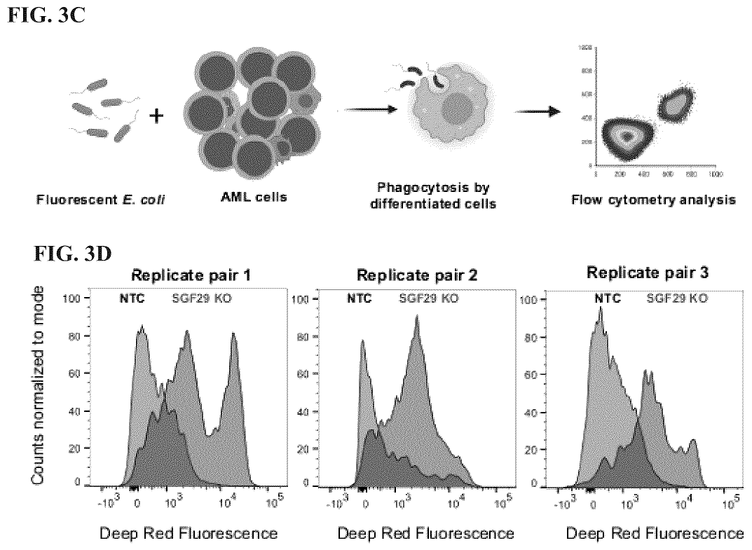

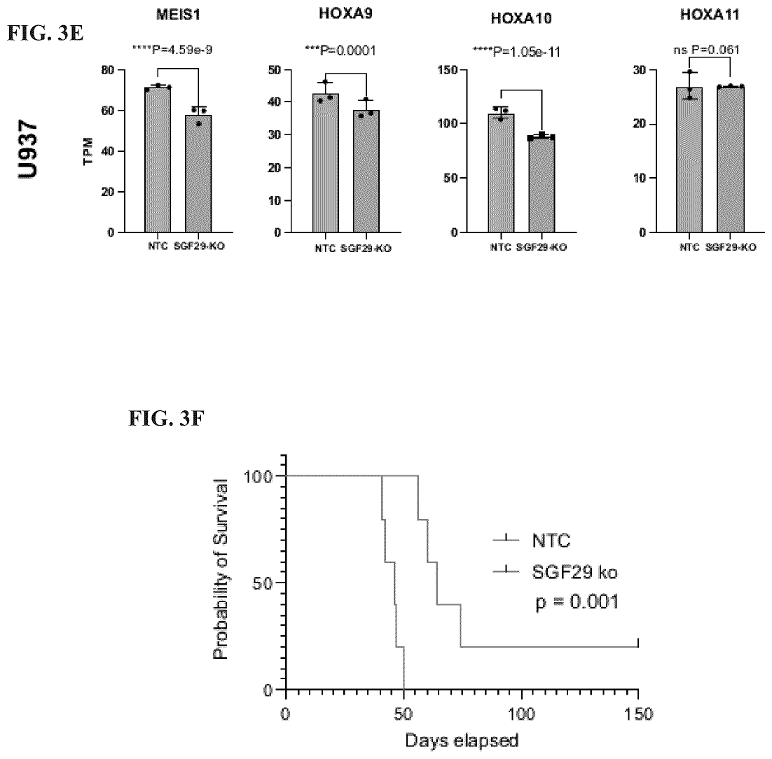

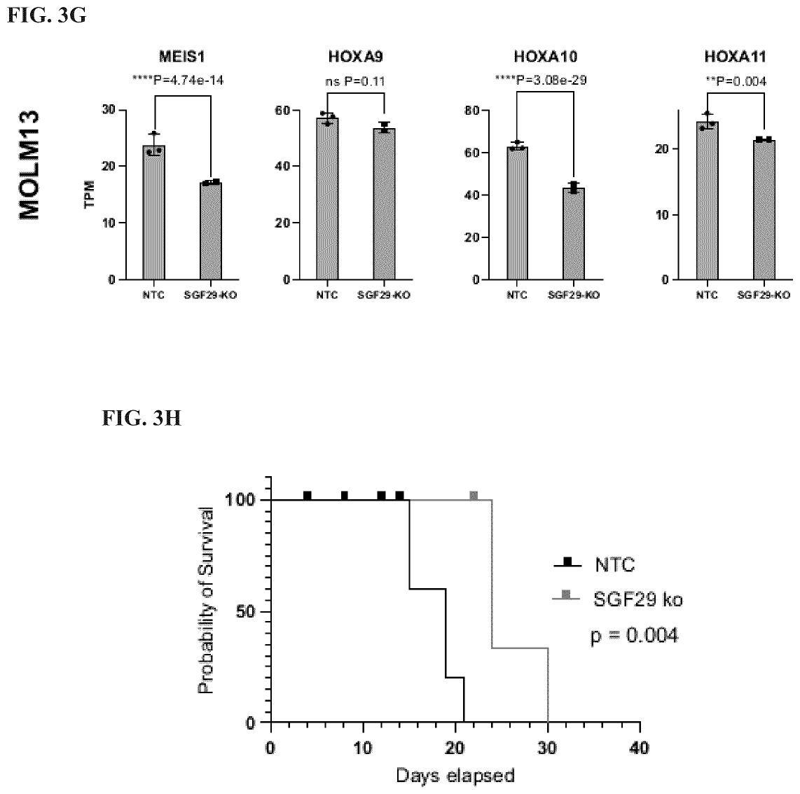

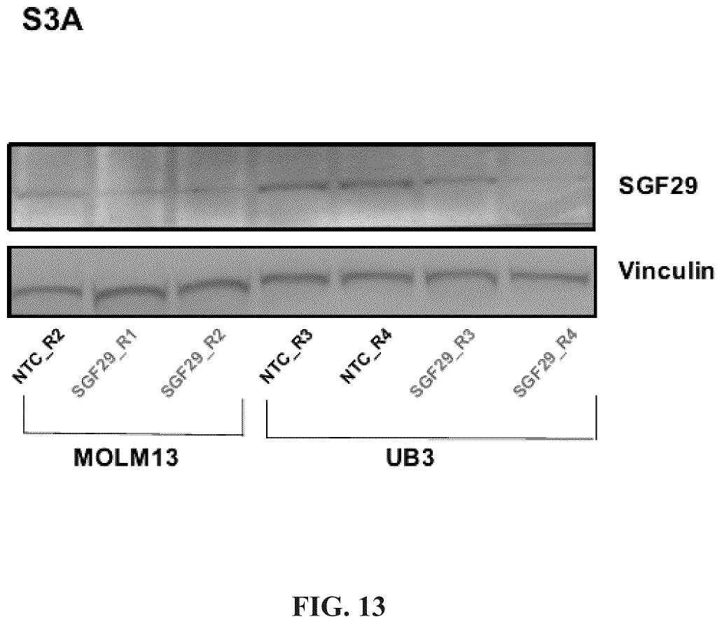

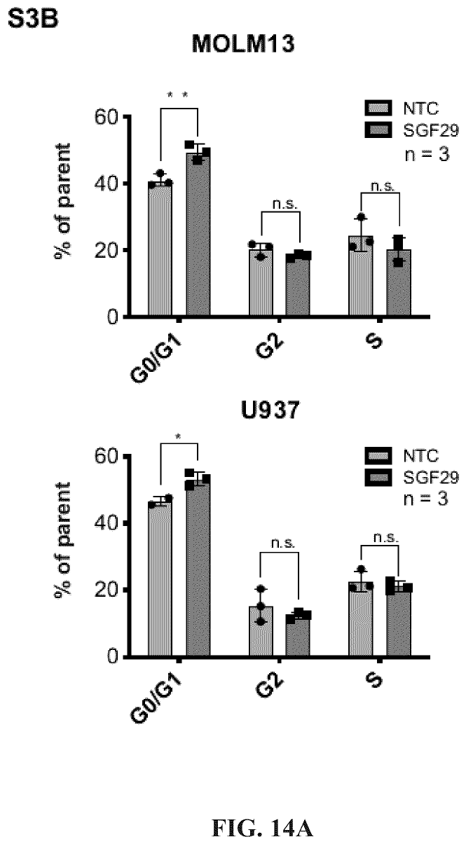

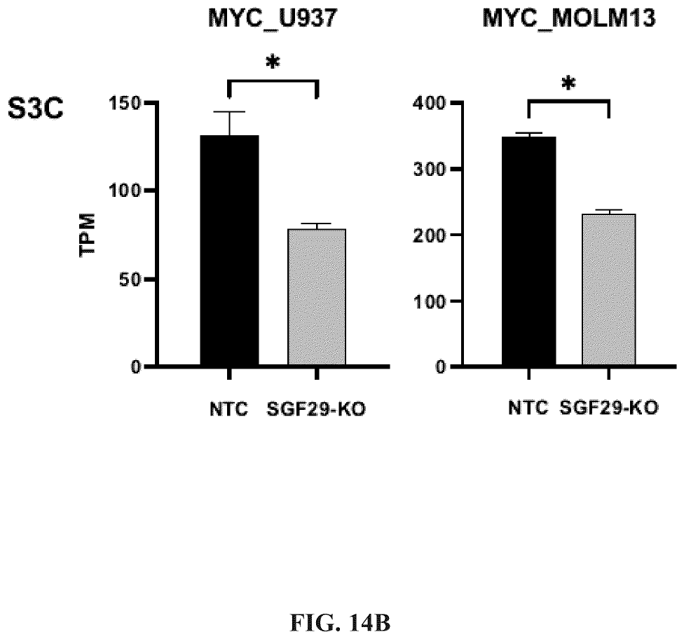

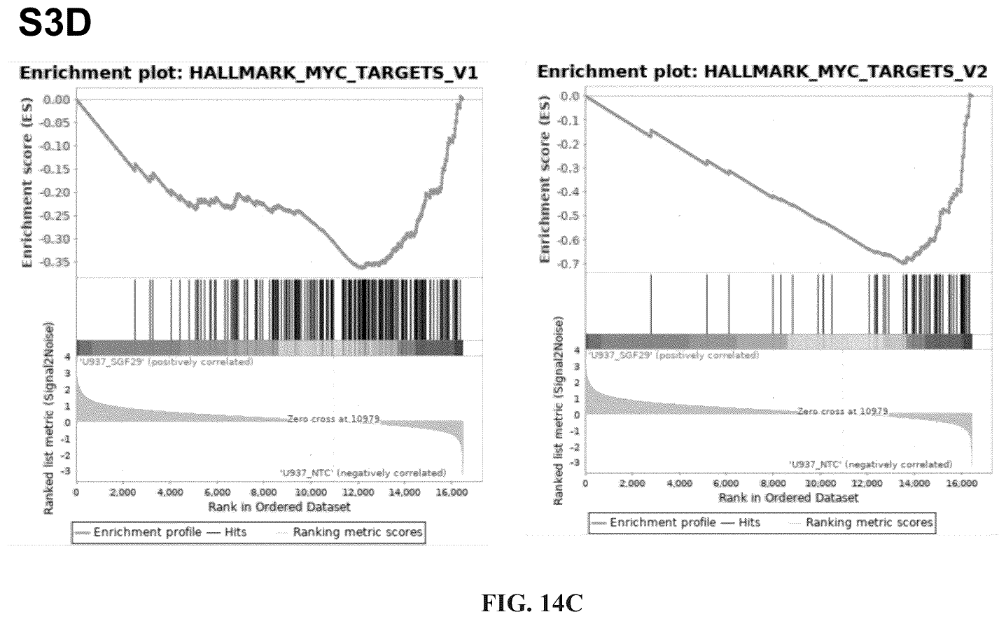

Transcriptomic, single sgRNA validation, and computational studies showed that SGF29 has one of the strongest dependencies from the hits across AML cell lines. Next, direct assessment of the impact of SGF29 deletion on human AML cell growth and leukemogenesis was performed. SGF29 inactivation using validated CRISPR sgRNAs (FIG. 13) led to significantly increased retention of the dye Cell Trace Violet (see Methods) in both U937 as well as MOLM13 AML cell lines, indicating diminished proliferation compared to cells treated with non-targeting controls (FIGS. 3A and 3B). Furthermore, the antiproliferative effects of SGF29 deletion were accompanied by a significant increase in the proportion of cells in the G0/G1 fraction compared to SGF29 wild-type cells (FIG. 14A). SGF29 deleted cells also showed a striking and significantly increased uptake of fluorescence-labeled heat-inactivated E coli bioparticles, indicating that SGF29 inactivation led to the myeloid differentiation of U937 cells (FIGS. 3C and 3D). Next, bulk RNA sequencing of MOLM13 and U937 cells with SGF29 CRISPR knockout compared to non-targeting control (NTC) cells was performed. RNA-seq analysis showed that SGF29 deletion significantly decreased the expression of several HOXA cluster genes in addition to MEIS1 in both MOLM13 and U937 cells (FIGS. 3E & 3G), confirming its role in HOX/MEIS regulation in diverse AML subtypes. Further, there was also a significant reduction in the expression of MYC (FIG. 14B) and the expression of MYC target genes was strikingly enriched in the non-targeting control AML cells compared to the SGF29 deleted counterparts, as assessed using gene-set enrichment analysis (GSEA) (28) (FIG. 14C). Given these strong effects of SGF29 deletion on leukemia oncogene expression and on AML cell growth, SGF29 deletion was tested and affected in vivo leukemogenesis of these human AML cell lines. In vivo studies using a cell line-derived xenograft model of leukemogenesis in NOD.Cg-Rag1^{tm1Mom} Il2rg^{tm1Wjl}//SzJ (NRG) mice, showed that SGF29 deletion led to a significantly increased disease latency in Cas9-expressing U937 as well as MOLM13 cell lines (FIG. 3F).

### Example 8. Genetic Sgf29 Inactivation Impairs the Clonogenicity of Transformed but not Normal Hematopoietic Progenitors

Next, given the role of SGF29 in influencing the transcription of self-renewal-associated genes, the effect of Sgf29 depletion on the clonogenic capability of myeloid bone marrow (BM) cells transformed by distinct AML driver oncoproteins was tested. Methylcellulose-based colony forming unit (CFU) assays on murine bone marrow hematopoietic stem and progenitor cells (HSPCs) transformed using the CALM-AF10, MLL-AF10 or MLL-AF9 oncofusion proteins were performed.

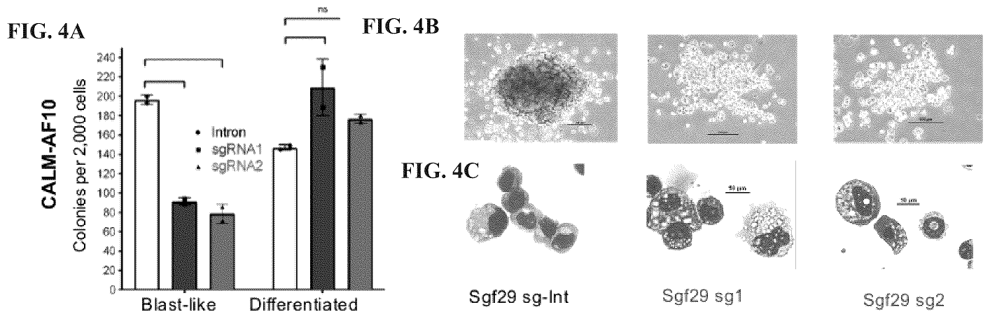

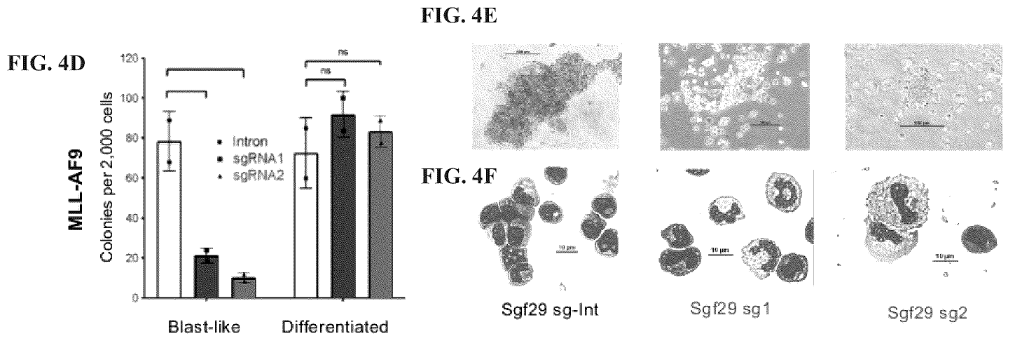

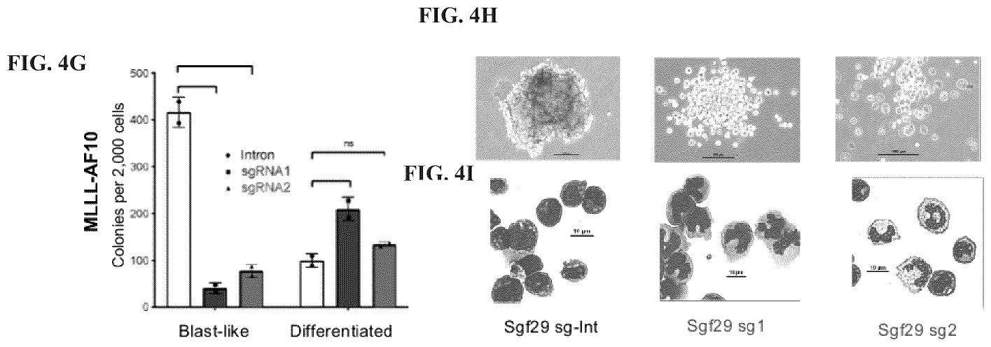

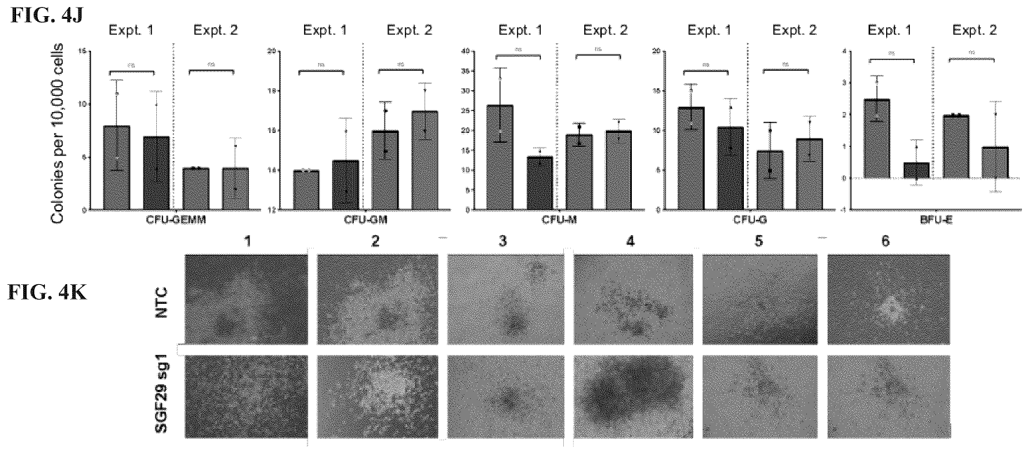

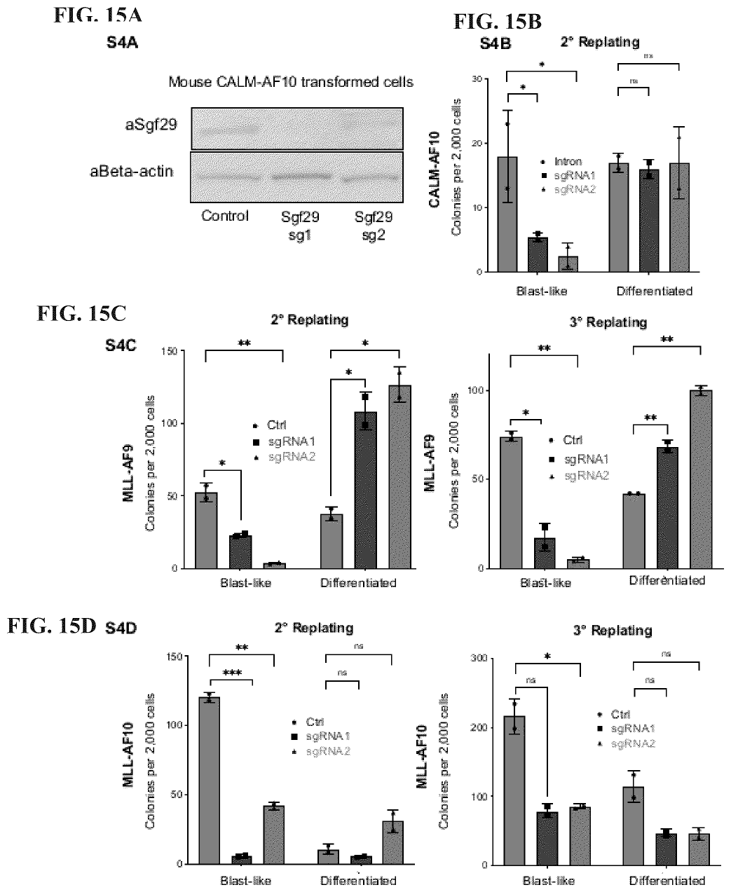

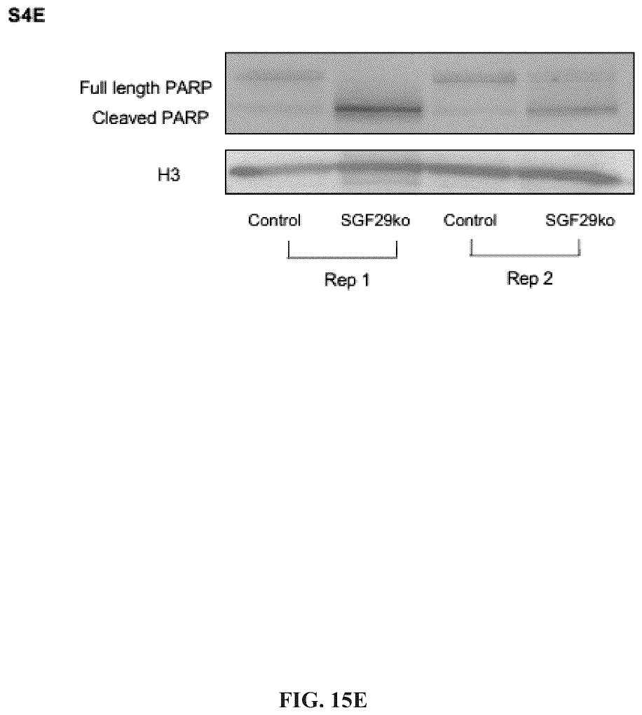

These studies showed that CRISPR knockout using two validated, (FIG. 15A) retrovirally expressed sgRNAs targeting Sgf29 coding exons led to a significant reduction in the number of colonies with a blast-like morphology in BM cells transformed with CALM-AF10 fusion oncoprotein, compared to the corresponding cells with an sgRNA targeting the Sgf29 intronic region (FIG. 4A-C). Surprisingly, while there was a dramatic decrease in the numbers of immature blast-like colonies, there were relatively modest effects on the number of differentiated colonies; thus, most colonies in the Sgf29−/− arm bore a differentiated morphology (FIG. 4B). The strong reduction in blast colony formation persisted for at least two rounds of replating (FIG. 15B). At the cellular level, in contrast to the immature, myeloid blast-like cellular morphology observed in Wright-Giemsa-stained cytospins of cells with wild-type Sgf29, the Sgf29 knockout cells resembled terminally differentiated, or differentiating myeloid lineage cells (FIG. 4C). Similar results were obtained from BM cells transformed using MLL-AF9 (FIG. 4D-F) and MLL-AF10 (FIG. 4G-I) fusion oncoproteins. Sgf29 deleted cells also showed increased apoptosis compared to Sgf29 wild-type cells as measured by immunoblotting for cleaved PARP (FIG. 15E). Next, the effect of Sgf29 deletion on the colony forming ability of normal HSPCs using the same sgRNAs that showed striking reductions in the formation of immature, blast-like colonies in myeloid transformed cells was tested. Surprisingly, in contrast to BM cells transformed using AML oncoproteins, CRISPR-mediated Sgf29 deletion in murine lineage negative, Sca-1 positive, Kit-positive (LSK) cells did not significantly alter either the number or type of colonies observed in CFU assays (FIG. 4J-K). Taken together, these data indicate that Sgf29 inactivation influences the clonogenicity and differentiation of cells transformed by diverse AIL oncoproteins, but not normal hematopoietic stem and progenitor cells (HSPCs).

### Example 9. The Tudor Domain of SGF29 is Essential for its Role in Myeloid Transformation

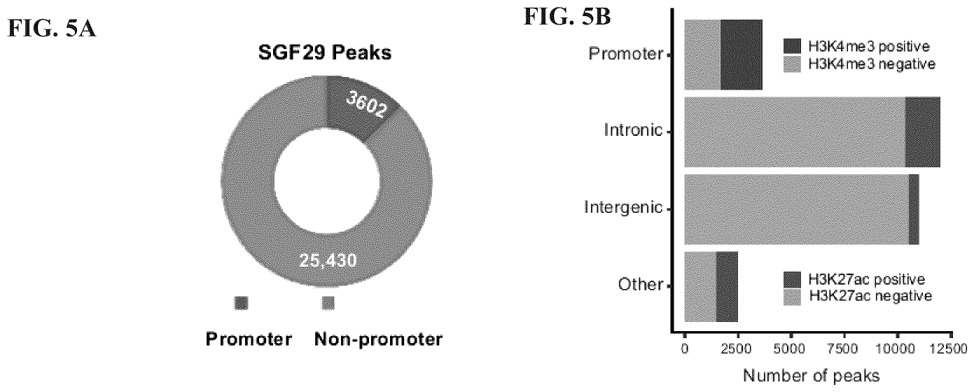

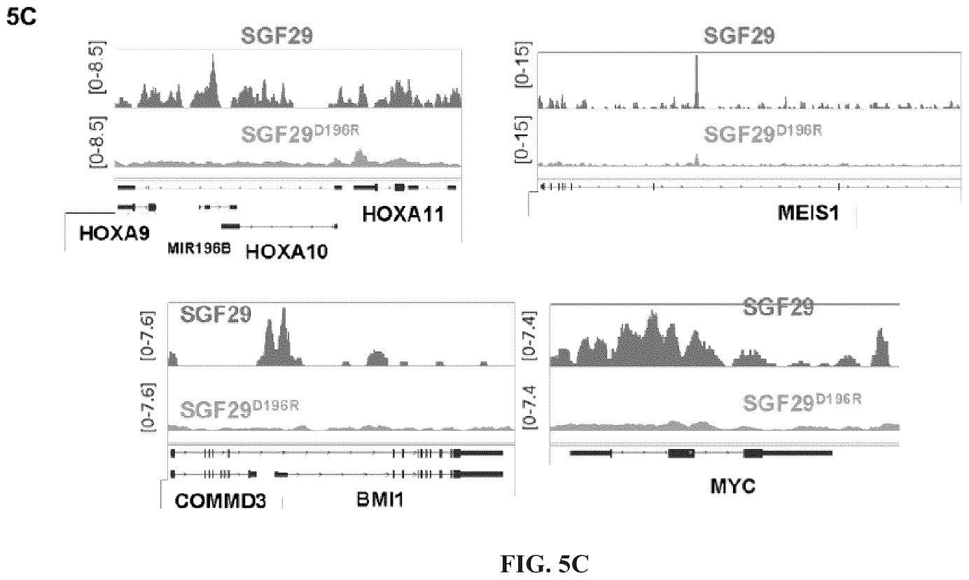

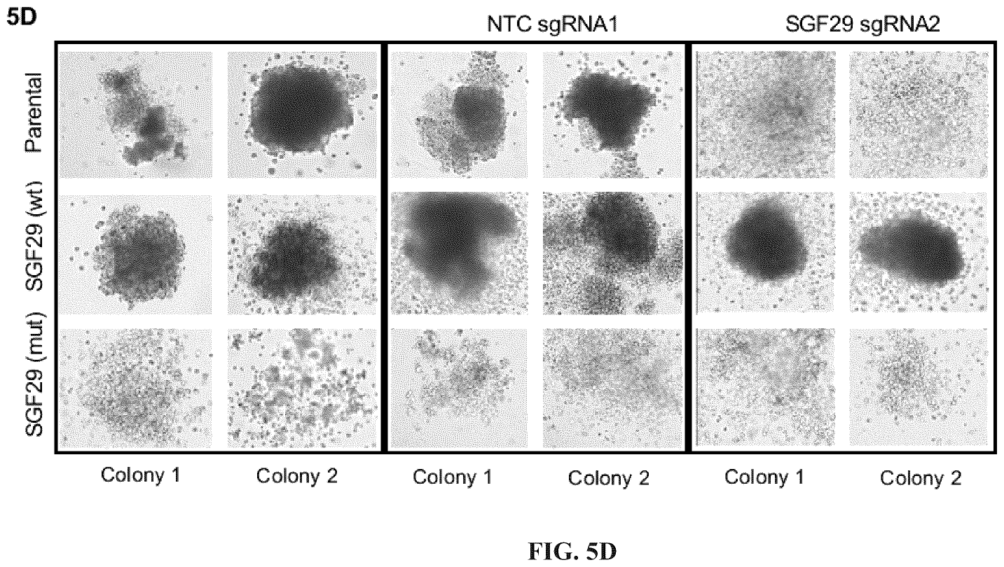

The SGF29 protein harbors a carboxy (C)-terminal tandem Tudor domain and this region is important in recognition of H3K4 di/trimethylated chromatin modifications and recruitment of KAT2A (GCN5)-containing chromatin-modifying complexes. In the epigenetic CRISPR screen, top-performing sgRNAs for SGF29 had homology to its Tudor domain, which confers H3K4me2/3 reader activity to SGF9. Thus, the Tudor domain is thought to be required for the transcriptional activation of AIL oncogenes including the HOX/MEIS genes, BMI1 and MYC. To assess the dependency of the Tudor domain for SGF29 localization in the HOX/MEIS loci, a Flag-tagged SGF29 gene and generated a stably transduced U937 cell line was cloned. Similarly, in order to test the effect of the SGF29 Tudor domain U937 cells expressing the SGF29^{D196R} mutant, which has been shown to disrupt H3K4me3 binding was generated. ChIP-sequencing using a Flag antibody in the Flag-SGF29 U937 cells showed that the protein occupied regions that were enriched for H3K4me3 marked active promoters and H3K27 acetylated enhancers (FIG. 5A&B). SGF29 occupied the promoter and/or enhancer regions of HOX/MEIS genes as well as other AML oncogenes whose expression was dependent on SGF29 (including BMI1 and MYC) (FIG. 5C). Surprisingly, analysis showed that in contrast to the wildtype SGF29, binding of the SGF29^{D196R} mutant to these gene loci was almost obliterated in SGF29^{D196R} mutant-expressing U937 cells (FIG. 5C). Next, assessment of whether the Tudor domain was important for the clonogenic capability of leukemia cells was commenced using the CFU assay. For this, the mouse MLL-AF9-transformed bone marrow cells which lose blast-like colony formation upon endogenous Sgf29 deletion was used. Using these cells, wildtype human SGF29 (impervious to the mouse anti-Sgf29 sgRNAs) or the SGF29D196R mutant counterpart using retroviral overexpression (FIG. 5D) was reinstated. Surprisingly, CFU assays demonstrated that ectopic overexpression of wildtype, but not mutant SGF29 could completely rescue the loss of blast-like CFUs upon endogenous Sgf29 deletion (FIG. 5D). Notably, ectopic expression of the SGF29^{D196R} mutant itself dramatically reduced the number of blast-like colonies even in MLL-AF9 cells with intact endogenous Sgf29 alleles, indicating that the SGF29^{D196R} mutant has dominant negative activity in the MLL-AF9 transformed cells (FIG. 5D).

### Example 10. Chromatin Proteomics Reveal the Role of SGF29 in Chromatin Localization of Proteins with Established Roles in AML Pathogenesis

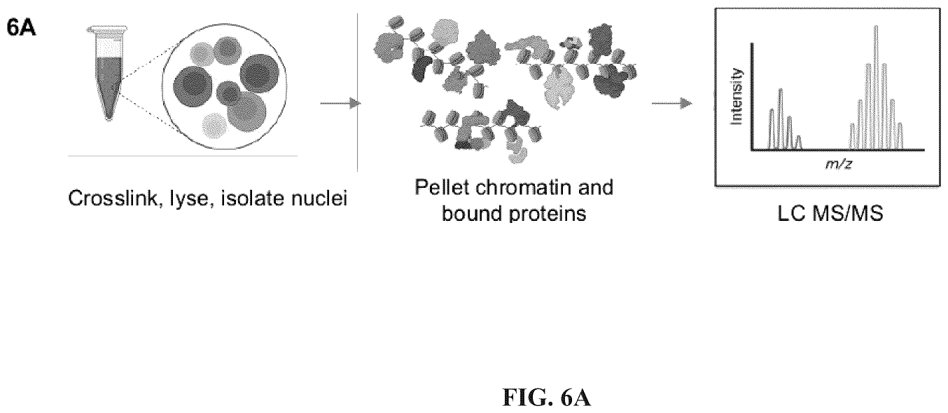

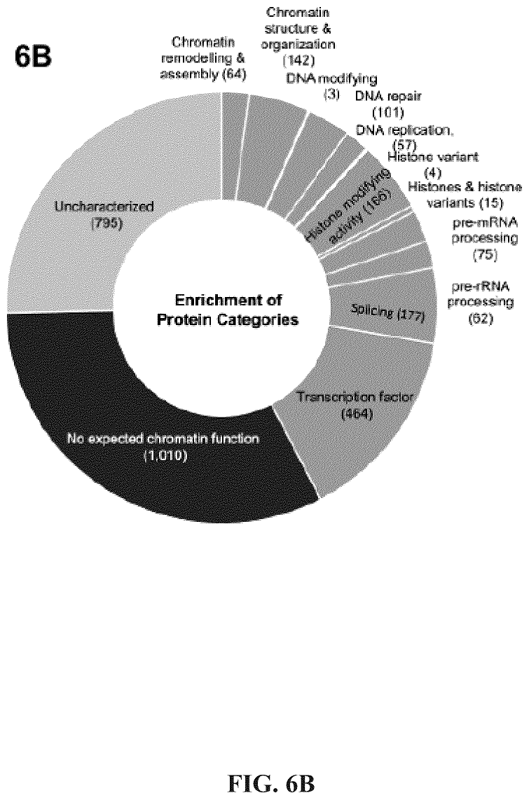

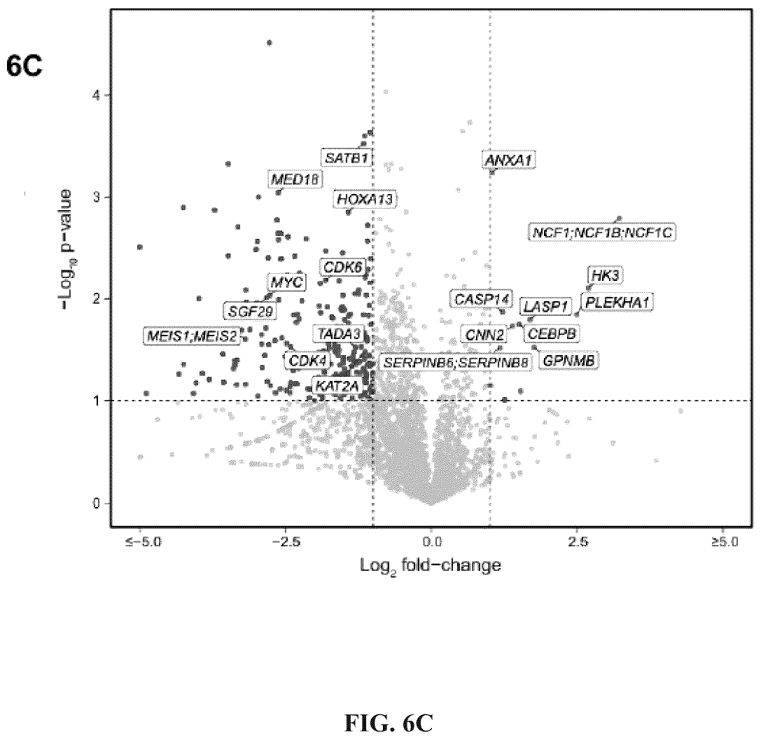

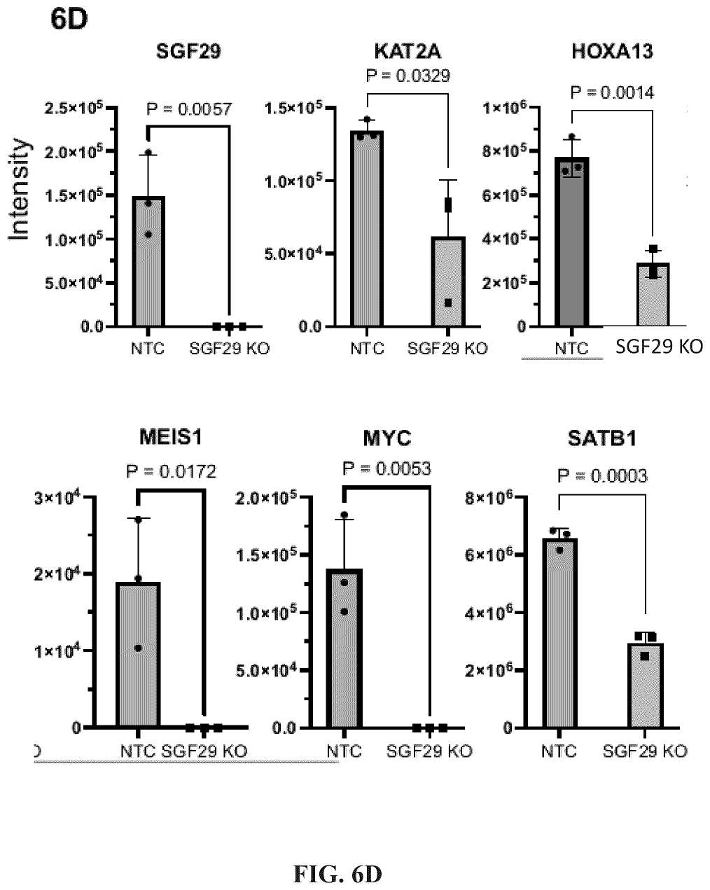

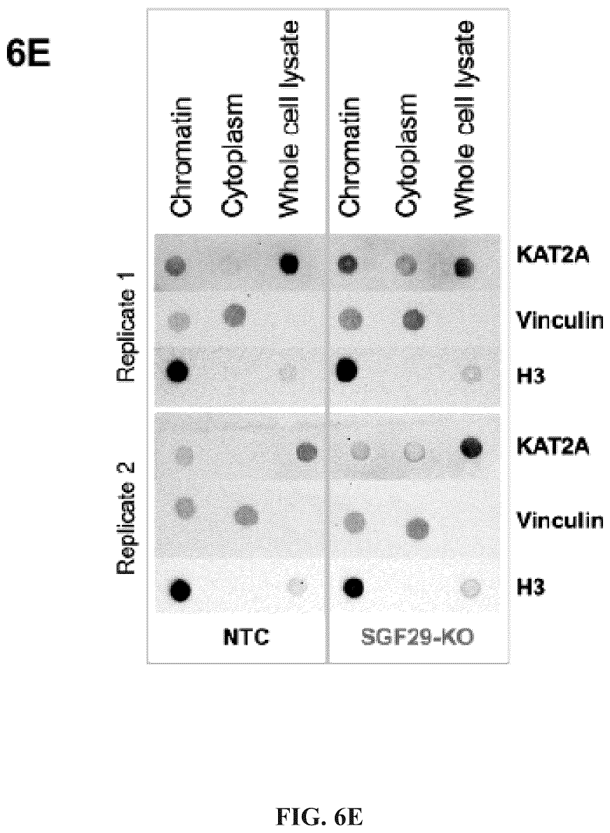

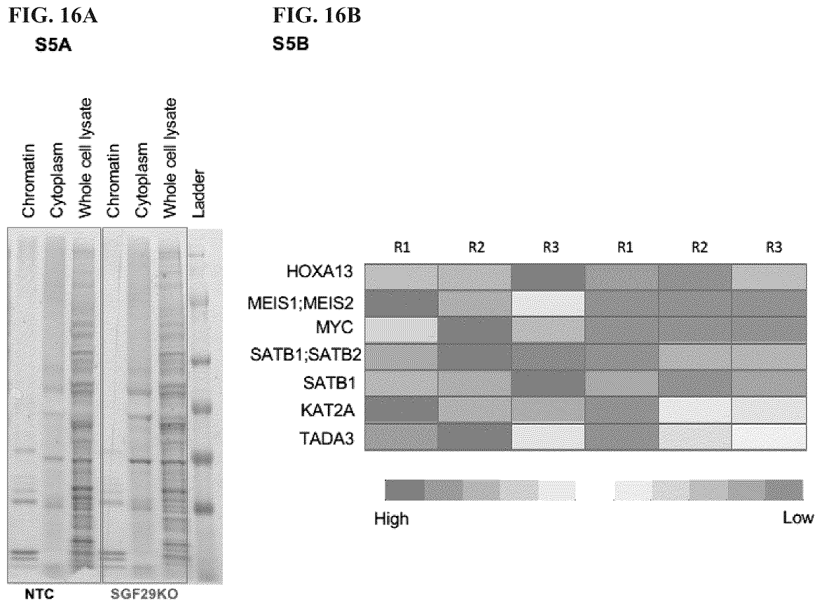

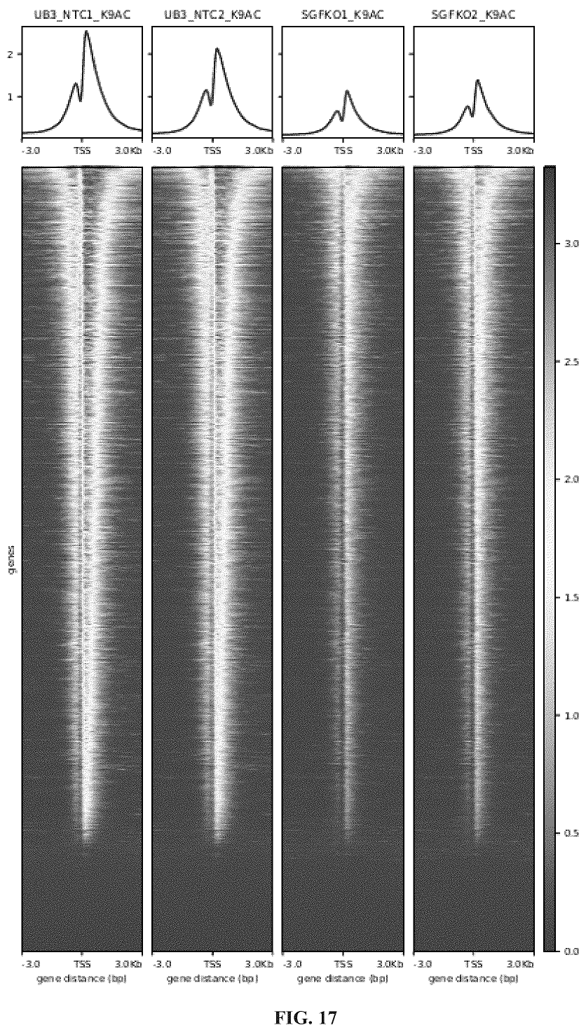

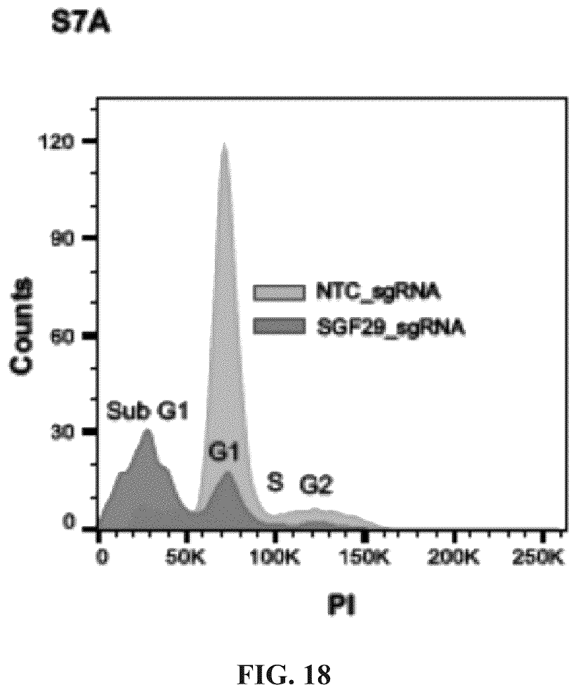

SGF29 is a participant of distinct chromatin complexes including the ADA2A-containing (ATAC) complex and the SPT3-TAF9-GCN5-acetyltransferase complex (STAGA) complex; the mammalian homolog of the yeast SAGA (SPT-ADA-GCN5-acetyltransferase complex). Both these complexes harbor the KAT2A (GCN5) and KAT2B (PCAF) acetyltransferases with histone acetylating activity and prominent roles in transcriptional activation. Thus, investigating which epigenetic regulators are evicted from chromatin upon SGF29 depletion in AML cells commenced. For this, chromatin enrichment proteomics (ChEP) (see FIG. 6A for schematic) commenced. This method allows an unbiased quantitative and qualitative assessment of the chromatin-associated proteome. ChEP purification yielded substantial enrichment of the chromatin/nuclear fraction as measured by the high proportional abundance of histones, histone modifying proteins, transcription factors and other chromatin and nuclear-associated proteins in the enriched fractions (FIG. 6B and FIG. 16A). SGF29 deletion in U937 cells significantly decreased the abundance of key AML oncoproteins, as measured by the intensity of chromatin-associated peptides by mass spectrometry. Specifically, the levels of HOXA13 and SATB1 were significantly reduced in the chromatin fraction of SGF29 deleted cells in contrast to their wildtype counterparts and the levels of MEIS1 and MYC were essentially undetectable in the chromatin fraction of SGF29 deleted cells in contrast to their SGF29 wildtype counterparts, where these proteins were highly abundant. (FIGS. 6C-D and FIG. 16B). A significant decrease in the abundance of CDK4 and CDK6 proteins, involved in cell cycle regulation and known to play prominent roles in the proliferation of cancer cells, particularly in AML was observed. Most importantly, ChEP data analysis demonstrated that SGF29 deletion led to a significant decrease in the chromatin abundance of key components of the STAGA complex, the histone acetyltransferase KAT2A, as well as the transcriptional adaptor protein TADA3 (FIG. 6C and FIG. 16B). These surprising results indicate that SGF29 deletion may lead to eviction of the STAGA complex from the chromatin fraction of U937 cells. In order to test this using an orthogonal approach, dot blots by immunoblotting using a KAT2A specific antibody in different cellular fractions in the SGF29 wildtype or CRISPR-deleted AML cells was performed. These studies demonstrated that while KAT2A could be found mostly in the chromatin fraction in wildtype U937 cells, SGF29 deletion indeed led to an increased abundance of KAT2A in the cytoplasmic fraction (FIG. 6E).

### Example 11. SGF29 Deletion Diminishes H3K9Ac on the Promoters of Key Leukemia Oncogenes

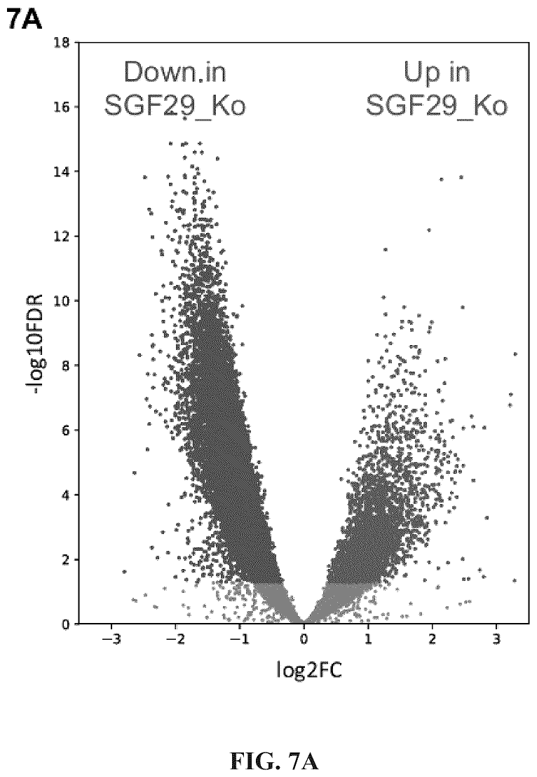

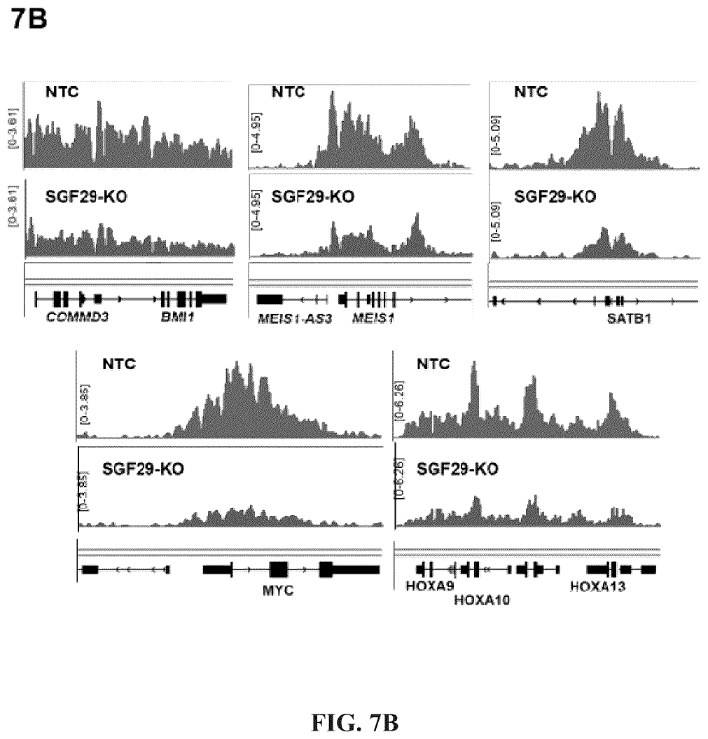

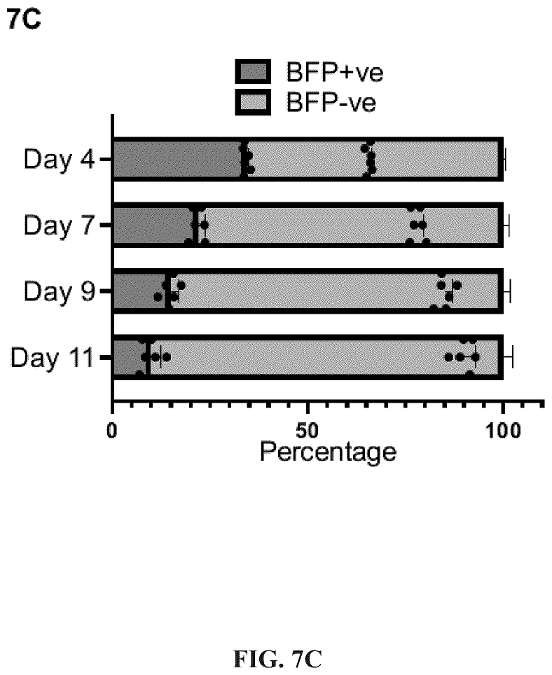

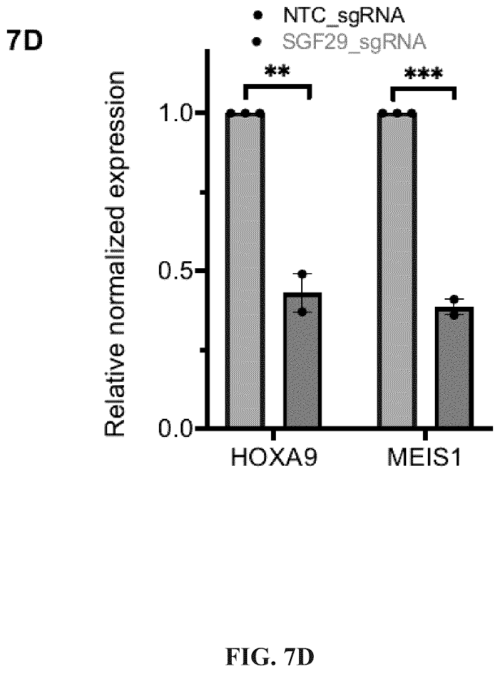

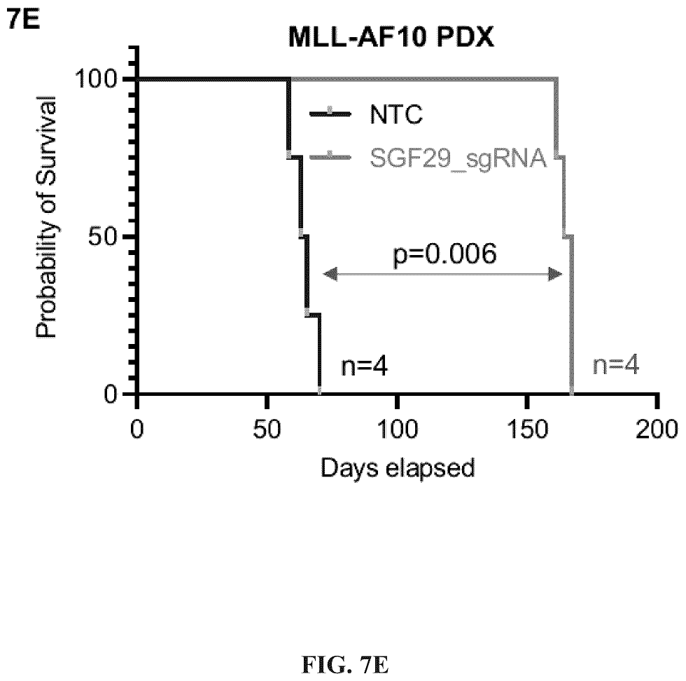

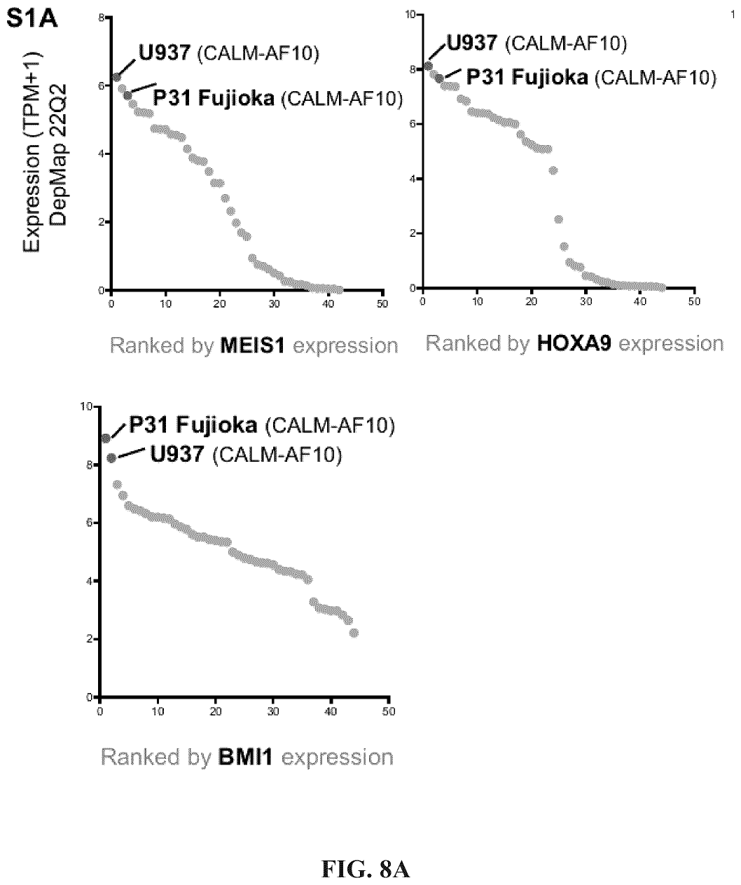

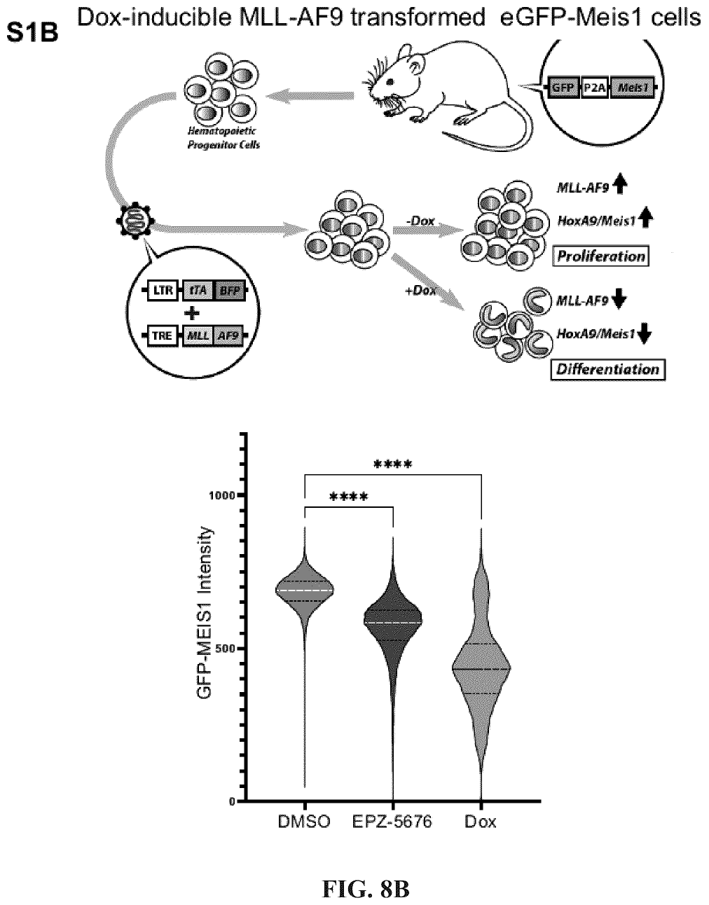

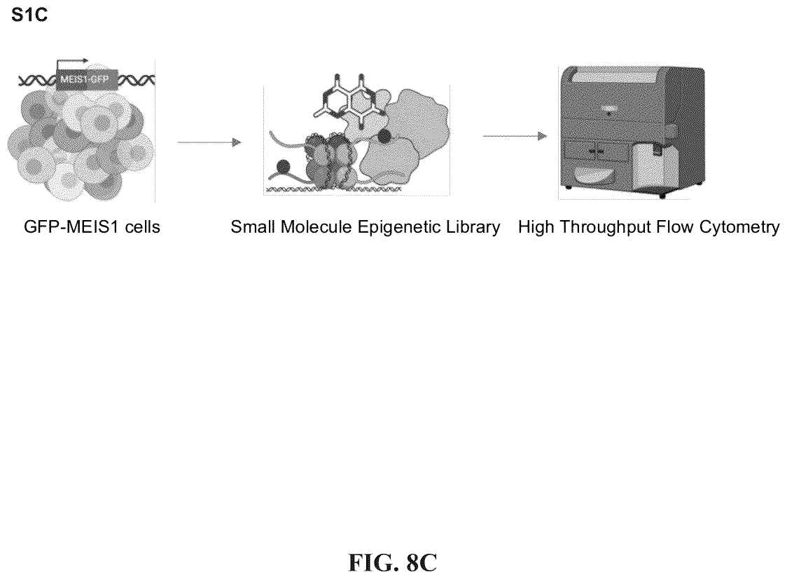

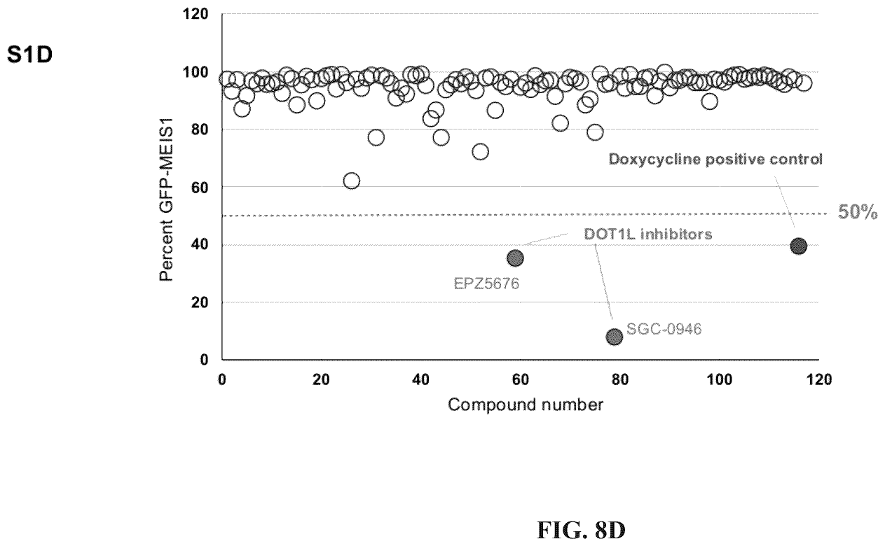

These results showing that SGF29 deletion decreased the abundance of the histone acetyltransferase KAT2A in the chromatin fraction led to the understanding that SGF29 is important for recruiting the chromatin activity of KAT2A on AML oncogene loci. KAT2A is the acetyltransferase subunit of the SAGA complex that plays important roles in leukemia and other cancers. ChIP-seq for H3K9Ac, the major histone modification deposited by the SAGA complex that is associated with transcriptional activation was tested to determine the activity of KAT2A on chromatin. Analysis of the ChIP-seq data showed that upon SGF29 deletion, there were striking changes in H3K9 acetylation, with 10,834 peaks showing reduced acetylation and 3,119 peaks showing increased acetylation in SGF29 knockout compared to wildtype U937 cells (FIG. 7A, and FIG. 17). Importantly, there was a pronounced decrease in acetylation levels at the promoter of several AML oncogenes that were also transcriptionally downregulated in the SGF29 knockout RNA-seq data. This included HOXA cluster genes, MYC, BMI1, and SATB1—all bona fide AML oncogenes with established roles in augmenting leukemic self-renewal (FIG. 7B).

### Example 12. SGF29 Deletion Impairs In Vitro and In Vivo Leukemogenesis in a Patient-Derived Xenograft Model of AML

The effect of SGF29 deletion in human AML patient cells was assessed. For this, AML393 cells were used, derived from an AML patient; these cells express split-Cas9 and can be used to perform gene-editing in a human AML sample. In AML393 cells, SGF29 knockout using the BFP-co-expressing sgRNAs led to a striking reduction in proliferative advantage in a competition assay compared to non SGF29-sgRNA expressing cells (FIG. 7C) and quantitative real-time PCR (q-RT-PCR) of SGF29-knockout compared to non-targeting control transduced AML393 cells showed a significant reduction of HOXA9 and MEIS1 transcripts in SGF29 deleted cells (FIG. 7D). The AML393 cells were injected with SGF29 knockout or non-targeting controls (NTC) into NRG mice and monitored engraftment of human CD45+ cells by flow-cytometric assessment of peripheral blood. Surprisingly, in comparison to the AML CDX models described in FIG. 3F&H, there were much more pronounced effects of SGF29 deletion in this human PDX model of AML. Whereas mice injected with NTC-targeting sgRNAs succumbed to AML with a median latency of 64 days, those injected with sgRNAs targeting SGF29 showed had a median disease latency of 165 days post injection (FIG. 7E). Taken together, these results demonstrate that SGF29 is important for sustaining critical transcriptional networks in leukemogenesis and may serve as an attractive therapeutic target in AML.

### Example 13. SGF29 Deletion Impacts Several Types of Cancers

Analysis of the DepMap Database shows that cell lines from several cancer types are sensitive to SGF29 deletion, including those from multiple myeloma, rhabdomyosarcoma, peripheral nervous system, breast cancer, lymphoma and skin cancer, in addition to leukemia cell lines. These result indicate that tumors of these lineages also depend on SGF29 and may benefit from SGF29 inhibition. Also, analysis of the DepMap Data showed that cell lines showing many AML subtypes are sensitive to SGF29 deletion. i.e: deletion of SGF29 using CRISPR affects the proliferation/fitness of AML cell lines with diverse mutational profiles. The highly sensitive AML subtypes include MLL/KMT2A rearranged AML cells (OCI-AML2, MOLM13 and SHI1), NPM1 mutant AML—OCI-AML3, AF10-rearranged cells—U937 and P31/Fuj, AML1-ETO positive cancers—SKNO1), PML-RARA positive leukemia—NB4. Many of these AML cell lines are P53 mutant (U937, MUTZ8, P31/FUJ) and still highly sensitive to SGF29 loss showing that SGF29 deletion impairs the growth of AML cells regardless of P53 mutational status.

### Example 14. SGF29 Deletion is Highly Selective for Leukemia Compared to Other Cancers

One of the challenges in developing epigenetic candidates as therapeutic targets is their potential for non-selective toxicity. One way of testing the selectivity of a candidate gene is to investigate the effects of its genetic deletion in a cancer subtype of interest compared to cancers of other lineages. For this, the dependency maps (DepMap) dataset was used, containing genome-scale functional genetic screens across cancer cell lines. The lineage specificity of the candidate genes' essentiality in leukemia was assessed by comparing the median CRISPR knockout fitness scores (Chronos) between leukemia (n=41) and non-leukemia (n=979) cell lines. In this analysis, depletion of SGF29, MLLT1/ENL, and DOT1L showed exceptionally high leukemia-selective fitness defects even more selective than KMT2A and MEN1 when compared to non-leukemia cells (Wilcox Test FDR q-values of 3.75E-08, 1.40E-06, and 2.25E-07, respectively). As leukemia cell lines in DepMap include both AML and non-AML cell lines, the specificity of SGF29 deletion effects for AML in the DepMap dataset was also tested. Again, SGF29, MLLT1/ENL, and DOT1L showed exceptional AML selectivity. Attention was focused on SGF29 due to its strong CRISPR deletion effects on U937, MOLM13, and MV-4-11 cell lines, high leukemia and AML selectivity in the DepMap data, strong effects on AML oncotranscriptome in CROP-seq studies, and most importantly—a yet uncharacterized role in AML. Interestingly, SGF29 transcripts were expressed at significantly higher levels in leukemia and AML cell lines compared to all other cancer cell lines in the DepMap data. This could help explain why SGF29 depletion has selective anti-proliferative effects in these lineages.

The examples and embodiments described herein are for illustrative purposes only and various modifications or changes suggested to persons skilled in the art are to be included within the spirit and purview of this application and scope of the appended claims.

1. A method of suppressing HOX/MEIS-activation in a cancer cell, comprising
(i) contacting the cancer cell with an inhibitor of SAGA-associated factor 29 (SGF29) protein, or
(ii) contacting the cancer cell with an inhibitory agent of SAGA-associated factor 29 (SGF29) protein synthesis.

2. The method of claim 1, wherein the inhibitor of SGF29 protein is a small molecule or an antibody, wherein the small molecule or antibody interacts with a Tudor domain of SGF29.

3. (canceled)

4. The method of claim 1, wherein the inhibitory agent of SGF29 protein synthesis is (i) a small interfering RNA (siRNA), (ii) a short hairpin RNA (shRNA), or (iii) an antisense oligonucleotide (ASO) that is complementary to an mRNA sequence.

5.-8. (canceled)

9. The method of claim 1, wherein the inhibitory agent of SGF29 protein synthesis is a CRISPR-Cas9 complex comprising (i) a Cas9 protein or a polynucleotide encoding the Cas9 protein, and (ii) a guide RNA or a polynucleotide encoding the guide RNA, wherein said guide RNA hybridizes with a nucleic acid sequence.

10. The method of claim 9, wherein the nucleic acid sequence is a DNA sequence.

11. (canceled)

12. (canceled)

13. The method of claim 1, wherein the cancer cell is an Acute Myeloid Leukemia (AML) cell, an Acute Lymphoblastic Leukemia (ALL) cell, a multiple myeloma cell, a rhabdomyosarcoma cell, a breast cancer cell, a lymphoma cell, or a skin cancer.

14. (canceled)

15. The method of claim 13, wherein the AML cell is CALM-AF10 fusion gene positive, MLL-AF9 fusion gene positive, an MLL/KMT2A rearranged AML cell, an NPM1 mutant AML cell, an AF10-rearranged cell, an AML1-ETO positive cancer cell, or a PML-RARA positive leukemia cell.

16. A method of treating cancer in a patient, comprising
(i) administering a therapeutically effective amount of an inhibitor of SAGA-associated factor 29 (SGF29) protein, or
(ii) administering a therapeutically effective amount of an inhibitory agent of SAGA-associated factor 29 (SGF29) protein synthesis, to the patient.

17. The method of claim 16, wherein the inhibitor of SGF29 protein is a small molecule or an antibody, wherein the small molecule or antibody interacts with a Tudor domain of SGF29.

18. (canceled)

19. The method of claim 16, wherein the inhibitory agent of SGF29 protein synthesis is (i) a small interfering RNA (siRNA), (ii) a short hairpin RNA (shRNA), or (iii) an antisense oligonucleotide (ASO) that is complementary to an mRNA sequence.

20.-23. (canceled)

24. The method of claim 16, wherein the inhibitory agent of SGF29 protein synthesis is a CRISPR-Cas9 complex comprising (i) a Cas9 protein or a polynucleotide encoding the Cas9 protein, and (ii) a guide RNA or a polynucleotide encoding the guide RNA, wherein the guide RNA hybridizes with a nucleic acid sequence.

25. The method of claim 24, wherein the nucleic acid sequence is a DNA sequence.

26. (canceled)

27. (canceled)

28. The method of claim 16, wherein the cancer cell is an Acute Myeloid Leukemia (AML) cell, an Acute Lymphoblastic Leukemia (ALL) cell, a multiple myeloma cell, a rhabdomyosarcoma cell, a breast cancer cell, a lymphoma cell, or a skin cancer.

29. The method of claim 16, wherein the cancer cell is CALM-AF10 fusion gene positive, MLL-AF9 fusion gene positive, an MLL/KMT2A rearranged AML cell, an NPM1 mutant AML cell, an AF10-rearranged cell, an AML1-ETO positive cancer cell, or a PML-RARA positive leukemia cell.

30.-60. (canceled)

61. A method for identifying a test agent for treating or preventing Acute Myeloid Leukemia (AML) or Acute Lymphoblastic Leukemia (ALL) in a patient having a CALM-AF10 fusion gene mutation in a cell, comprising:
a. contacting a SAGA-Associated Factor 29 (SGF29) protein with the test agent;
b. determining whether the test agent interacts with a SGF29 protein; and
c. subjecting the test agent to an in vitro, an ex vivo, or an in vivo test, wherein the test agent that interacts with a SGF29 protein reduces or prevents cell proliferation.

62. The method of claim 61, wherein determining whether the test agent interacts with SGF29 protein comprising measuring a Green Fluorescent Protein (GFP) signal, wherein determining whether the test agent interacts with SGF29 protein comprises measuring growth of cells over time, wherein determining whether the test agent interacts with SGF29 protein comprises measuring leukemic engraftment in one or more mice treated with the test agent, or wherein determining whether the test agent interacts with SGF29 protein comprises measuring a level of at least one protein, wherein at least one protein is selected from the group consisting of Homeobox A13 (HOXA13), SATB Homeobox 1 (SATB1), Meis Homeobox 1 (MEIS1) and MYC proteins.

63. The method of claim 62, wherein the GFP signal shows reduction over an increasing dose of the test agent.

64. (canceled)

65. The method of claim 62, wherein there is a decrease of growth of cells over time relative to nontreatment.

66. (canceled)

67. The method of claim 62, wherein there is an abolition or decrease of leukemic engraftment in at least one or more mice treated with the test agent relative to nontreatment.

68. (canceled)

69. The method of claim 62, wherein there is an abolition or decrease of protein level relative to nontreatment.

70.-108. (canceled)
